(19) United States
(12) Patent Application Publication
(10) Pub. No.: US 2025/0078261 A1
(43) Pub. Date: Mar. 6, 2025

(54) OBJECT DETECTION AND MEASUREMENTS IN MULTIMODAL IMAGING

(71) Applicant: SpectraWAVE, Inc., Bedford, MA (US)

(72) Inventors: Damon T. DePaoli, Cambridge, MA (US); David A. Vader, Concord, MA (US); Eman Namati, Concord, MA (US)

(21) Appl. No.: 18/728,072

(22) PCT Filed: Jan. 20, 2023
(86) PCT No.: PCT/US23/11267

§ 371 Date: Jul. 10, 2024

Related U.S. Application Data
(60) Provisional application No. 63/301,486, filed on Jan. 20, 2022.

(51) Int. Cl. G06T 7/00; A61B 5/00; G06V 10/77

(52) U.S. Cl. CPC G06T 7/0012; A61B 5/0084; A61B 5/7267; G06T 7/97; G06V 10/7715; G06V 2201/03

(57) ABSTRACT

Systems and methods for detecting features, outputting feature representations, determining measurements, and combinations thereof using machine-learned algorithms are provided. In some embodiments, first sample data from a first characterization modality and second sample data from a second characterization modality are provided to a machine-learned algorithm. Improved feature detection, feature representation, and measurement may be realized by using multimodal data with a machine-learned algorithm. In some embodiments, a first characterization modality is an interferometric modality and a second characterization modality is a spectroscopic modality. A first characterization modality may be optical coherence tomography and a second characterization modality may be a diffuse spectroscopy modality, such as near-infrared spectroscopy. Sample data may be intraluminal and/or vascular data useful in characterizing a vascular system of a subject, such as a human.

### PRIORITY APPLICATION

This application claims the benefit of U.S. Provisional Patent Application No. 63/301,486, filed on Jan. 20, 2022, the disclosure of which is hereby incorporated by reference herein in its entirety.

### TECHNICAL FIELD

This disclosure relates generally to methods of detecting and characterizing objects within images of bodily lumens using more than one disparate data source.

### BACKGROUND

Multimodal imaging systems have been used to characterize lumens, such as arteries. An example of such a system combines spectroscopy and interferometric imaging. Generally, feature detection and measurements, where performed, use data from one mode or the other. For example, spectroscopy data provide information about the presence or absence of plaque in a location and interferometric data provide structural information. While images may be spatially registered to present information to a clinician, it continues to be difficult, in some cases, to make determinations about the presence or absence of certain features of interest (e.g., to differentiate between plaque types) and/or to make measurements on those features.

### SUMMARY

Aspects of the disclosure relate to data from disparate characterization modalities being used as inputs to a machine-learned algorithm (e.g., a multimodal machine-learned feature detector), for example for intraluminal image segmentation. First sample data may be from a first characterization modality. Second sample data may be from a second characterization modality. The first sample data and second sample data may be pre-processed or processed (e.g., ran through a feature extractor) prior to being provided to a machine-learned algorithm (e.g., as input). A machine-learned algorithm may then output detected feature(s) (of interest) (e.g., as selected by a user, during training and/or when running the algorithm with test data), transformed (e.g., enhanced) feature representation(s), measurement(s) on feature representation(s) for detected feature(s), or a combination thereof. A first data source, for example acquired with a first characterization subsystem, may be interferometric. For example, first sample data may be or include coherence-gated, depth resolved imaging (e.g., as is the case with optical coherence tomography (OCT)). A second data source, for example acquired with a second characterization subsystem, may be spectroscopy-based. For example, second sample data may be or include molecular information (e.g., via diffuse spectroscopy, such as near-infrared spectroscopy (NIRS)). An interferometric data source and a spectroscopy-based data source may be together used as inputs to a machine-learned algorithm, for example a multimodal machine-learned feature detector. The multiple datasets provide disparate sources of information but can improve image segmentation of the depth resolved imaging modality, for example by using machined-learned feature detectors.

Sample characterization (e.g., of materials) can be performed using electromagnetic radiation (e.g., light). In some instances, characterization can be improved if more than one method of characterization is performed (e.g., multimodal characterization). However, there exist challenges in multimodal characterization. For instance, building a system capable of multimodal characterization is one challenge. Another is maximizing the utility of the multimodal information gathered. Each modality may provide similar, or disparate, sources of data that can be either exploited in parallel or in sequence to take advantage of the other, at least in some aspect. For example, one data source may encode spatial information and provide a depth resolved image (e.g. optical coherence tomography (OCT)) while another data source may encode molecular information based on a spectrum from a single point (e.g., diffuse reflectance spectroscopy (DRS), such as near-infrared spectroscopy (NIRS)). Each data source may detect light from different sample volumes, for example due to optical properties of the sample being characterized and/or wavelength(s) of light being used by the different characterization modalities. Furthermore, while each data source may be processed and used for the detection of unique outputs, each may provide information that can strengthen the other's detection algorithms. Hand-crafted, sequential approaches that provide compensatory information between multiple algorithms rarely take full advantage of all the information stored in each data source. By using modern feature detection algorithms (e.g., machine learning, such as deep learning and/or neural networks) with inputs from each data source, higher order information can be exploited. Disclosed herein are, inter alia, methods to optimize the detection of intraluminal features using multimodal systems that provide disparate characterization data sources, by providing both data sources into a machine-learned algorithm (e.g., feature detector) having access to both modalities simultaneously.

Intraluminal imaging of tissues can take advantage of, and is affected by, many optical phenomena. For instance, the scattering properties of tissue can affect the amount and direction of light scattering that occurs when tissue is illuminated. Rayleigh and Mie scattering are sources of elastic scattering causing a photon to change direction without a change in energy. Elastic scattering can provide information on index of refraction, density of molecules, orientation of molecules, and/or the structure of a sample, among other things. Raman scattering is a source of inelastic scattering changing a photon's energy and direction of propagation. With inelastic scattering, the amount of energy change is dependent on the vibration of a unique molecular bond and this energy shift (e.g., wavelength shift) can be used to probe the molecular composition of the tissue. Inelastic scattering is a much less efficient optical phenomena than elastic scattering, occurring in approximately 1 in every 10,000,000 photons.

The absorption properties of a tissue can affect the amount of energy at a given wavelength that is deposited (e.g., absorbed) in the tissue when it is illuminated. Due to the wavelength dependent nature of molecular absorption, absorption properties of a tissue can provide information on its molecular composition. Importantly, absorption is a highly efficient process. When light is absorbed, the primary energy transfer is from light to heat; however, other optical interactions may also occur. For example, some tissues have fluorescent molecules that can be excited when the energy of the illumination wavelength matches a specific band gap of the molecule, causing it to absorb the light and subsequently reemit it at a lower energy. Fluorescence can therefore be used to probe the molecular composition of a tissue based on the amount and wavelength dependent shape of re-emitted light spectrum. Fluorescence can be innate to a native tissue molecule, known as auto-fluorescence. Fluorescence can also be exploited by binding (e.g., labeling, tagging) a native tissue molecule with a fluorescent probe (e.g., tag, molecule, label) (e.g., covalently binding fluorescent dyes to biomolecules).

Scattering and absorption occur simultaneously and are sometimes difficult to characterize separately. There are modalities, however, that depend more so on one effect than the other. For instance, interferometric imaging (e.g., OCT) depends on the depth dependent scatterers in the tissue to produce an image, while diffuse spectroscopy (e.g., fluorescence, Raman, diffuse reflectance spectroscopy (DRS)) depends on both scattering and absorption. Therefore, OCT and diffuse spectroscopy share fundamental information, but are not identical. For example, illumination at a focused point in tissue using OCT creates a depth resolved line of an image, discerning its structure. On the other hand, diffuse spectroscopy is an integration of the scattered light within a light interrogation volume and can provide a wavelength dependent spectrum describing the molecular composition of the tissue but generally provides no depth-resolved data. In a probe orientation, OCT generally measures backscattered light through a singlemode waveguide as coherence is important in performing interferometric imaging. Diffuse spectroscopy (e.g., fluorescence, Raman, DRS) on the other hand often uses multimode waveguides, as collection efficiency is a primary parameter to optimize.

The efficiency of optical phenomena is a key attribute in developing multimodal systems. Some modalities, such as Raman spectroscopy are inefficient and require long integration times (e.g., at least 100 ms). Other modalities, like OCT, can image at exceptionally high sampling rates (e.g. 1-10 μs, e.g., 5 μs), due to the efficiency of the optical process. This difference is especially important in some clinical contexts (e.g., intravascular imaging), where imaging may be performed at high rotational speeds (e.g., at least 10,000 rpm) over long distances (e.g., at least 100 mm) in short periods of time (e.g., 1-3 s, e.g., 2 s), for example, to image a lumen (e.g., a coronary artery) during temporarily flushing of the blood out of the imaging path (e.g., using saline or radiopaque contrast). While one approach could be to image with one modality and then to subsequently image with another, an optimal approach would be to perform the imaging at approximately the same time (e.g., interwoven, in parallel, simultaneously) to minimize acquisition time and registrations artifacts (e.g., from heart movement) between the two modalities. Therefore, to begin with, imaging systems should carefully optimize multimodal optics, detection scheme(s) and acquisition timing, in order to provide high-fidelity co-registered data.

Optical designs that optimize multiple modalities can be further complicated by the geometry of the optics. In diffuse spectroscopy scenarios, such as DRS, wherein the same wavelengths carry the detected signal as the illumination light, specular reflection may hinder the valuable signal which originates from light that has interacted with the sub-surface tissue. In other scenarios, such as fluorescence imaging, wherein the wavelengths of detected light differ from the illumination wavelengths, filters can be used to limit illumination light and therefore maximize collection efficiency. OCT measures backscattered light, but not in a diffuse manner and requires focused illumination and detection. A multimodal system may therefore measure light originating from different volumes of tissue, by optimizing the geometry of the optics.

The ability to perform multimodal imaging at high speeds has only recently been made possible at commercially viable costs. The combination of high-speed light sources, detectors and rotational systems now allow the acquisition of co-registered multimodal information from small-form-factor rotational probes. With the advancement of acquisition hardware, the focus has shifted to innovation of data analytic methods capable of intelligently optimizing the information from co-registered intraluminal datasets (e.g., spectroscopic and tomographic information).

Imaging modalities often provide complementary information for feature detection purposes (e.g., segmentation and/or classification). For instance, segmenting an object in an OCT image often relies on contrast and morphology of a structure. It may be possible to segment the object, but the classification of the object may be difficult for similarly scattering objects with similar shapes (e.g., coronary plaques) (e.g., fibrous vs. lipid rich plaques). Diffuse spectroscopy, which can detect molecular composition (e.g., using near-infrared spectroscopy (NIRS)), and angularly inform on the type of tissue within a segmented image. As disclosed herein, these disparate sources of information can be combined to improve detection (e.g., classification) accuracy and specificity of features within an OCT image.

Multimodal imaging algorithms can also improve segmentation for a single modality. For instance, some objects in an OCT image may not be completely resolved (e.g., due artifacts such as to non-uniform rotational distortion (NURD)) (e.g., inadequate axial resolution) (e.g., inadequate lateral resolution) (e.g., attenuation) leading to uncertain (e.g., low confidence) regions of segmentation on class probability maps. In such cases, a diffuse spectroscopy input may be able to inform a spatial segmentation algorithm, improving confidence of segmentation for certain segmentation classes. For example, a stent which is poorly resolved by OCT, leaving only a visible shadow, may still have high diffuse reflectance at a given position, due to the different volumes tissue being imaged by two modalities.

Multimodal imaging algorithms can also improve regression. For example, diffuse spectroscopy may be capable of detecting and/or classifying an object based on a spectrum, but may have challenges quantifying a molecule (e.g., lipid) due to the geometric optical factor (e.g., distance of the object from the imaging system) (e.g., the thickness of the object), or other confounding objects in the line of sight of the object to quantify. In such cases, information from a structural imaging modality, such as OCT, may allow an algorithm to limit the effects of confounding factors and improve quantification.

Machine-learned algorithms (e.g., multimodal imaging algorithms) can also transform (e.g., enhance) the output of a modality. For example, an OCT image may have artifacts present, or may attenuate rapidly. A machine-learned algorithm that has information on tissue fluorescence may allow enhancement of an OCT image by increasing low contrast areas or decreasing high contrast areas. As another example, an enhanced image may become an important input into another algorithm for feature detection and/or measurement. In some embodiments, a machine-learned algorithm may transform (e.g., enhance) one or more feature representations of one or more features of interest, for example that are detected by a machine-learned algorithm.

Machine-learned algorithms (e.g., multimodal imaging algorithms) can also transform a modality's visual representation. For example, DRS can provide information on the “color” of a tissue. A multimodal algorithm that has information from both an OCT image and a DRS spectrum could therefore aid a color transformation. For example, an image transforming algorithm may be trained on co-registered white light (e.g., visible spectrum) images and OCT images, transforming OCT images to pseudo-colored images. An input into such an exemplary image transforming algorithm may allow improved image transformation to a colored image (e.g., indicating plaque with one or more particular colors).

The present disclosure recognizes that improved (e.g., higher sensitivity, specificity, accuracy, fidelity) detection [e.g., semantic segmentation, 1D (e.g. line-based) segmentation, bounding box segmentation], transformation (e.g., enhancement), and measurement (e.g., quantification, regression) algorithms (e.g., separate or multi-functional algorithms) may require multi-modal and sometimes disparate sources of data to significantly boost performance of automated results and enable effective clinical decision making. Indeed, a clinician does not necessarily have the time or expertise to understand and correlate multi-data sources such as spectral and/or structural information; however, a machine can learn to do so with high performance, speed and accuracy using machine-learning techniques disclosed herein. As such, the current disclosure describes and enables, inter alia, multimodal feature detection (e.g., segmentation) via a combination of disparate data sources of overlapping and complimentary information, including structure and spectroscopy data, that can improve on single modality predecessors (e.g., in the context of intraluminal imaging).

In some aspects, the present disclosure is directed to a method for detecting a feature of interest. The method may include receiving, by a processor of a computing device, first sample data from a first characterization modality and second sample data from a second characterization modality. The method may further include detecting, by the processor, a feature of interest [e.g., a structure external or internal to a subject (e.g., a physiological structure)] by providing the first sample data and the second sample to a machine-learned algorithm. The method may further include outputting (e.g., from the algorithm), by the processor, a feature representation of the feature of interest that is oriented with respect to the first characterization modality.

In some embodiments, the machine-learned algorithm has been trained to detect two or more features of interest. In some embodiments, the first sample data and the second sample data are both from intraluminal characterization modalities. In some embodiments, the first characterization modality is an interferometry modality (e.g., OCT) and the second characterization modality is an intensity measurement (e.g., a fluorescence modality). In some embodiments, the first characterization modality is a depth-dependent imaging modality (e.g., OCT) and the second characterization modality is a wavelength-dependent measurement modality (e.g., NIRS). In some embodiments, one or both of the first characterization modality and the second characterization modality are processed (e.g., formatted) prior to being input to the machine-learned algorithm. In some embodiments, one or both of the first characterization modality and the second characterization modality have been registered (e.g., to one another) prior to being input to the machine-learned algorithm.

In some embodiments, the method includes registering, by the processor, one or both of the first sample data and the second sample data (e.g., to one another) prior to inputting the first sample data and the second sample data to the machine-learned algorithm. In some embodiments, the machine-learned algorithm has been trained to detect features using labels from either only the first characterization modality or the second characterization modality. In some embodiments, the machine-learned algorithm outputs detected features in reference to the first characterization modality (e.g., only the first characterization modality). In some embodiments, the first sample data are generated from the first characterization modality detected at a first region having a first tissue volume within a bodily lumen and the second sample data are generated from the second characterization modality detected at a second region having a second volume within a bodily lumen.

In some embodiments, an intraluminal characterization volume of each modality does not completely overlap. In some embodiments, the first region and the second region do not completely overlap. In some embodiments, the first sample data are generated from detection with the first characterization modality at a time t₁ and the second sample data are generated from detection with the second characterization modality at time t₂, where t₂−t₁<1 ms. In some embodiments, the first sample data and the second sample data are combined into a combined sample data and the combined sample data are input into the machine-learned algorithm during the detecting step. In some embodiments, the combining of the first sample data and the second sample data includes (e.g., consists of) appending the first sample data to the second sample data. In some embodiments, the appending includes merging the first sample data and the second sample data together.

In some embodiments, the machine-learned algorithm has multiple stages where data can be input. In some embodiments, information from the first characterization modality and from the second characterization modality are input to the machine-learned algorithm as two unique inputs at different stages. In some embodiments, the first sample data and the second sample data are separately input to the machine-learned algorithm at different ones of the multiple stages. In some embodiments, each sample data undergoes feature extraction, and outputs of the feature extraction are used as inputs to the machine-learned algorithm.

In some embodiments, the method includes: inputting, by the processor, the first sample data and the second sample data to one or more feature extractors; and generating, by the processor, outputs from the one or more feature extractors, wherein the detecting comprises inputting the outputs from the one or more feature extractors into the machine-learned algorithm.

In some embodiments, the method includes segmenting and classifying, by the processor, one or more foreign objects (e.g., one or more fiber optics, one or more sheaths, one or more stent struts, one or more balloons). In some embodiments, the method includes segmenting and classifying, by the processor, the one or more foreign objects with the machine-learned algorithm (e.g., using the first sample data and the second sample data as inputs to the algorithm) (e.g., wherein the detecting step comprises the segmenting and classifying). In some embodiments, the method includes segmenting and classifying, by the processor, one or more vascular structures (e.g., lumen, intima, medial, external elastic membrane, branching). In some embodiments, the method includes segmenting and classifying, by the processor, the one or more vascular structures with the machine-learned algorithm (e.g., using the first sample data and the second sample data as inputs to the algorithm) (e.g., wherein the detecting step comprises the segmenting and classifying).

In some embodiments, the method includes segmenting and classifying, by the processor, plaque morphology based on contents of the plaque (e.g., calcium, macrophages, lipids, collagen or fibrous tissue). In some embodiments, the method includes segmenting and classifying, by the processor, the plaque morphology with the machine-learned algorithm (e.g., using the first sample data and the second sample data as inputs to the algorithm) (e.g., wherein the detecting step comprises the segmenting and classifying). In some embodiments, the method includes segmenting and classifying, by the processor, one or more necrotic cores or thin-cap fibroatheroma (TCFA). In some embodiments, the method includes segmenting and classifying, by the processor, the one or more necrotic cores or TCFA with the machine-learned algorithm (e.g., using the first sample data and the second sample data as inputs to the algorithm) (e.g., wherein the detecting step comprises the segmenting and classifying).

In some embodiments, the method includes detecting (e.g., segmenting and classifying), by the processor, one or more arterial pathologies. In some embodiments, the method includes detecting (e.g., segmenting and classifying), by the processor, the one or more arterial pathologies with the machine-learned algorithm (e.g., using the first sample data and the second sample data as inputs to the algorithm) (e.g., wherein the detecting step comprises the segmenting and classifying).

In some embodiments, the method includes detecting (e.g., segmenting and classifying), by the processor, one or more spectroscopy-sensitive markers. In some embodiments, the method includes detecting (e.g., segmenting and classifying), by the processor, the one or more spectroscopy-sensitive markers (e.g., one or more fiducials) with the machine-learned algorithm (e.g., using the first sample data and/or the second sample data as inputs to the algorithm) (e.g., wherein the detecting step comprises the segmenting and classifying). In some embodiments, the method includes adjusting (e.g., correcting), by the processor, an image (e.g., an image produced by an interference technique, e.g., an OCT image) based on the detection of the one or more spectroscopy-sensitive markers (e.g., thereby reducing effect of non-uniform rotational distortions of a probe (e.g., imaging catheter) observable in the image).

In some embodiments, the feature of interest includes (e.g., is) one or more foreign objects, one or more vascular structures, plaque morphology, one or more arterial pathologies, one or more necrotic cores or thin-cap fibroatheroma, or any combination thereof.

In some embodiments, detecting, by the processor, the feature of interest includes segmenting and classifying the feature of interest. In some embodiments, the method includes determining, by the processor, one or more measurements based on the feature of interest (e.g., using the machine-learned algorithm). In some embodiments, the one or more measurements include a geometric measurement (e.g., angle, thickness, distance). In some embodiments, the one or more measurements includes an image-based measurement (e.g., contrast, brightness, histogram).

In some embodiments, the method includes determining, by the processor, a bad frame, insufficient blood flushing, contrast injection detection, or a combination thereof with the machine-learned algorithm (e.g., using the first sample data and the second sample data as inputs to the algorithm).

In some embodiments, the method includes automatically (e.g., by the processor) initiating pullback of an imaging catheter and/or scanning of a probe based on the feature of interest detected with the machine-learned algorithm. In some embodiments, the method includes determining, by the processor, an optical probe break based on the feature of interest detected with the machine-learned algorithm.

In some embodiments, the method includes detecting, by the processor, poor transmission based on the feature of interest detected with the machine-learned algorithm. In some embodiments, the method includes correcting, by the processor, an image (e.g., an image produced by an interference technique, e.g., an OCT image) based on the feature of interest detected with the machine-learned algorithm (e.g., thereby reducing effects of non-uniform rotational distortions of a probe (e.g., imaging catheter) observable in the image).

In some embodiments, the method includes generating the first sample data using the first characterization modality and the second sample data using the second characterization modality. In some embodiments, generating the first sample data and the second sample data includes performing a catheter pullback.

In some embodiments, the method includes enhancing, by the processor, the feature representation with respect to the first characterization modality based on the second sample data and/or with respect to the second characterization modality based on the first sample data (e.g., automatically with the machine-learned algorithm) (e.g., wherein the enhanced feature representation is output from the machine-learned algorithm).

In some embodiments, the feature representation is registered to the first sample data, the second sample data, or both the first sample data and the second sample data. In some embodiments, the method includes outputting (e.g., displaying), by the processor, the feature representation overlaid over an image (e.g., OCT image) derived from the first sample data or the second sample data.

In some embodiments, the method is performed after pullback of a catheter with which the first sample data and the second sample data are acquired (e.g., automatically upon completion of the pullback). In some embodiments, the feature representation of the feature of interest that is oriented also with respect to the second characterization modality.

In some embodiments, the outputting comprises displaying (e.g., on a display included in a system, such as a catheter system). In some embodiments, the outputting is via one or more graphical user interfaces (GUIs). In some embodiments, the outputting comprises storing (e.g., on a non-transitory computer readable medium).

In some aspects, the present disclosure is directed to a multimodal system for feature detection, the system including: a processor; and a non-transitory computer readable medium having instructions stored thereon that when executed by the processor automatically upon initiation of a characterization session, cause the processor to: receive, by the processor a first sample data from a first characterization modality and second sample data from a second characterization modality; detect, by the processor, a feature of interest by providing the first sample data and the second sample to a machine-learned algorithm; and output, by the processor, a feature representation of the feature of interest that is oriented with respect to the first characterization modality.

In some embodiments, the system further includes a display. In some embodiments, the instructions, when executed by the processor automatically upon initiation of the characterization session, cause the processor to output the feature representation with the display. In some embodiments, the system further includes a first characterization subsystem for the first characterization modality and a second characterization subsystem for the second characterization modality. In some embodiments, the instructions, when executed by the processor automatically upon initiation of the characterization session, cause the processor to perform a method described herein. In some embodiments, the system is a catheter system. In some embodiments, the system includes a probe that is operable to collect sample data for one or more characterization modalities [e.g., two characterization modalities (e.g., an interferometric modality (e.g., OCT) and a spectroscopic modality (e.g., NIRS))]. The probe may be sized and shaped to collect and transmit light from inside a body lumen (e.g., artery) to one or more detectors. The one or more detectors may be included in the system.

In some aspects, the present disclosure is directed to a method for measuring a feature of interest, the method including: receiving, by a processor of a computing device, first sample data from a first characterization modality and second sample data from a second characterization modality; detecting, by the processor, a feature of interest by providing the first sample data and the second sample data to a machine-learned algorithm; and determining, by the processor, one or more measurements of a feature representation of the feature of interest.

In some embodiments, the method further includes displaying, by the processor, the measurement on a display. In some embodiments, the first sample data and the second sample data are both from intraluminal characterization modalities. In some embodiments, the measurement includes a geometric measurement (e.g., angle, thickness, distance, depth). In some embodiments, the measurement includes an image-based measurement (e.g., contrast, brightness, histogram). In some embodiments, the measurement quantifies an aspect of a foreign object within a lumen (e.g., one or more fiber optics, one or more sheaths, one or more stent struts, one or more balloons). In some embodiments, the measurement relates to positioning of a foreign object within a lumen (e.g., stent placement within an artery). In some embodiments, the measurement quantifies an aspect of a vascular structure (e.g., of a lumen, an intima, a medial, an external elastic membrane, branching). In some embodiments, the measurement quantifies an aspect of plaque morphology (e.g., quantity of calcium, macrophage, lipid, fibrous tissue, or necrotic core within an area). In some embodiments, the measurement quantifies risk associated with a detected plaque (e.g., a detected TCFA). In some embodiments, the measurement includes a cap thickness over a lipid pool or necrotic core. In some embodiments, the measurement includes a plaque burden or lipid core burden (e.g., max burden over a distance). In some embodiments, the measurement includes plaque vulnerability. In some embodiments, the measurement includes a calcium measurement (e.g., arc, thickness, extent, area, volume, ratio of calcium to other). In some embodiments, the measurement includes a lipid measurement (e.g., arc, thickness, extent, area, volume, ratio of lipid to other). In some embodiments, the measurement includes stent malapposition, stent length, or stent location planning.

In some embodiments, the method includes automatically determining, by the processor, (e.g., with one or more machine-learned algorithms, e.g., the machine-learned algorithm) a location for a stent placement based on the one or more measurements (e.g., by optimization). In some embodiments, the one or more measurements comprises lumen area. In some embodiments, the measurement comprises a measurement on an external elastic membrane or external elastic lamina. In some embodiments, the machine-learned algorithm outputs the measurement.

In some embodiments, the method includes generating the first sample data using the first characterization modality and the second sample data using the second characterization modality. In some embodiments, generating the first sample data and the second sample data includes performing a catheter pullback.

In some aspects, the present disclosure is directed to a method for enhancing data acquired from a bodily lumen, the method including: receiving, by a processor of a computing device, first sample data from a first characterization modality and second sample data from a second characterization modality; detecting, by the processor, a feature of interest by providing the first sample data and the second sample to a machine-learned algorithm; and outputting from the algorithm, by the processor, a transformed representation of either the first sample data or the second sample data, or both, based on the detected feature.

In some embodiments, the method includes displaying (e.g., by the processor) the transformed representation (e.g., wherein the outputting comprises displaying the transformed representation). In some embodiments, the method includes inputting, by the processor, the transformed representation into another machine-learned algorithm for feature detection. In some embodiments, the method includes detecting, by the processor, a feature of interest with the machine-learned algorithm for feature detection based on the transformed representation input. In some embodiments, the transformed representation is an enhanced OCT image. In some embodiments, the transformed representation corrects for non-uniform rotational distortions of a probe using the detected feature. In some embodiments, the transformed representation is enhanced reflectance data. In some embodiments, the transformed representation is enhanced spectroscopy data. In some embodiments, the transformed representation is in a new image space or color scheme. In some embodiments, the method includes determining, by the processor, a specular versus diffuse reflection ratio based on one of the first sample data and the second sample data (e.g., with the machine-learned algorithm) and improving or enhancing attenuation correction in the transformed representation, wherein the transformed representation is of either the other of the first sample data and the second sample data. In some embodiments, the transformed representation is an attenuation corrected representation.

In some aspects, the present disclosure is directed to a method for detecting a feature of interest, the method including: receiving, by a processor of a computing device, first sample data from a first characterization modality and second sample data from a second characterization modality; and detecting, by the processor, one or more features of interest by providing the first sample data and the second sample to a machine-learned algorithm. In some embodiments, the method includes automatically (e.g., by the processor) initiating (i) pullback of an imaging catheter and/or (ii) scanning of a probe based on the one or more features of interest detected with the machine-learned algorithm.

In some aspects, the present disclosure is directed to a method for compensating for non-uniform rotational distortions (NURD), the method including: receiving, by a processor of a computing device, first sample data from a first characterization modality and second sample data from a second characterization modality; evaluating, by the processor, NURD by providing at least one of the first sample data and the second sample to a machine-learned algorithm; and correcting, by the processor, (e.g., with the machine-learned algorithm) at least one of the first sample data and the second sample data based on the evaluating to accommodate for NURD.

In some embodiments, the evaluating includes providing only the first sample data to the machine-learned algorithm and the correcting is of the second sample data. In some embodiments, the first sample data and/or the second sample data are an image.

In some aspects, the present disclosure is directed to a method for determining improved physiological measurements, the method including: receiving, by a processor, first sample data from a first characterization modality; receiving, by the processor, information about a feature of interest (e.g., a location and/or composition of the feature of interest), wherein at least a portion of the first sample data corresponds to the feature of interest (e.g., a feature representation of the feature of interest is comprised in the first sample data); and determining, by the processor, a physiological measurement using the first sample data and the information.

In some embodiments, the feature of interest is a plaque or a curvature of a vessel (e.g., an artery). In some embodiments, the physiological measurement corresponds to a flow, a pressure drop, or a resistance for a vascular structure (e.g., artery) (e.g., wherein the physiological measurement is a measurement of flow rate, fractional flow reserve, pressure drop, absolute or relative coronary flow (CF), fractional flow reserve (FFR), instantaneous wave free ratio/resting full cycle ratio (iFR/RFR), index of microcirculatory resistance (IMR), hyperemic microvascular resistance (HMR), hyperemic stenosis resistance (HSR), coronary flow reserve (CFR), or a combination thereof).

In some embodiments, the first characterization modality is an interferometric modality (e.g., OCT). In some embodiments, the method includes determining, by the processor, the location and/or composition of the feature of interest using second sample data from a second characterization modality (e.g., and also the first sample data). In some embodiments, the second characterization modality is a spectroscopic modality (e.g., NIRS). In some embodiments, the method includes determining, by the processor, the information about the feature of interest using the first sample data.

In some embodiments, the method includes determining, by the processor, the information about the feature of interest using a machine-learned algorithm [e.g., by providing the first sample data (e.g., and/or the second sample data) to the machine-learned algorithm]. In some embodiments, the method includes detecting, by the processor, the feature of interest using a (e.g., the) machine-learned algorithm [e.g., by providing the first sample data (e.g., and/or the second sample data) to the machine-learned algorithm].

In some aspects, the present disclosure is directed to a method for making physiological measurements, the method including: receiving, by a processor of a computing device, first sample data from a first characterization modality and second sample data from a second characterization modality; and determining, by the processor, a physiological measurement by providing the first sample data and the second sample data to a machine-learned algorithm.

In some aspects, the present disclosure is directed to a method of training a machine-learned algorithm, the method including: providing, by a processor of a computing device, training data to a machine-learning algorithm, wherein the training data is labelled with training labels that have been derived from data from a second characterization modality different from the first characterization modality.

In some embodiments, the first characterization modality is an interferometric modality (e.g., OCT) and the second characterization modality is a spectroscopic modality (e.g., NIRS). In some embodiments, the training data does not comprise data from the second characterization modality.

In some aspects, the present disclosure is directed to a method for detecting a feature of interest and/or determining a measurement thereof, the method including: receiving, by a processor of a computing device, first sample data from a first characterization modality [e.g., an interferometric modality (e.g., OCT)]; detecting, by the processor, a feature of interest by providing the first sample data to a machine-learned algorithm that has been trained on training data from the first characterization modality, the training data labelled with training labels derived from data from a second characterization modality [e.g., a spectroscopic modality (e.g., NIRS)]; and outputting (e.g., from the algorithm), by the processor, (i) a feature representation of the feature of interest that is oriented with respect to at least the first characterization modality, (ii) one or more measurements (e.g., of the feature representation and/or comprising a physiological measurement), or (iii) both (i) and (ii).

In some embodiments, the machine-learned algorithm does not accept data from the second characterization modality as input. In some embodiments, the machine-learned algorithm accepts data from no other characterization modality than the first characterization modality as input. In some embodiments, the method includes outputting, by the processor, the feature representation and/or at least one measurement of the feature representation, wherein the feature of interest is a plaque or portion thereof (e.g., lipid core of the plaque).

In some aspects, the present disclosure is directed to a system, the system including: a processor; and a non-transitory computer readable medium having instructions stored thereon that when executed by the processor (e.g., automatically upon initiation of a characterization session), cause the processor to perform a method disclosed herein.

In some embodiments, the system further includes a display. In some embodiments, the instructions, when executed by the processor automatically upon initiation of the characterization session, cause the processor to output the feature representation with the display. In some embodiments, the system further includes a first characterization subsystem for the first characterization modality and a second characterization subsystem for the second characterization modality. In some embodiments, the instructions, when executed by the processor automatically upon initiation of the characterization session, cause the processor to perform a method described herein. In some embodiments, the system is a catheter system. In some embodiments, the system includes a probe that is operable to collect sample data for one or more characterization modalities [e.g., two characterization modalities (e.g., an interferometric modality (e.g., OCT) and a spectroscopic modality (e.g., NIRS))]. The probe may be sized and shaped to collect and transmit light from inside a body lumen (e.g., artery) to one or more detectors. The one or more detectors may be included in the system.

In some aspects, the present disclosure is directed to a non-transitory computer readable medium having instructions stored thereon that when executed by a processor, cause the processor to perform a method disclosed herein.

Any two or more of the features described in this specification, including in this summary section, may be combined to form implementations of the disclosure, whether specifically expressly described as a separate combination in this specification or not.

### BRIEF DESCRIPTION OF THE DRAWINGS

Drawings are presented herein for illustration purposes, not for limitation. Drawings are not necessarily to scale. The foregoing and other objects, aspects, features, and advantages of the disclosure will become more apparent and may be better understood by referring to the following description together with the accompanying figures, in which:

FIG. 1 illustrates a diagram of a multimodal characterization system in accordance with illustrative embodiments of the present disclosure;

FIG. 2 is a flowchart of a method of using a multimodal feature detection system in accordance with illustrative embodiments of the present disclosure;

FIG. 3 is a flowchart of a method of using a multimodal feature enhancer system in accordance with illustrative embodiments of the present disclosure;

FIG. 4 is a flowchart of a method of using a multimodal feature detection and measurement system in accordance with illustrative embodiments of the present disclosure;

FIG. 5 is a flowchart of a method of using a multimodal feature detection system in accordance with illustrative embodiments of the present disclosure;

FIG. 6A illustrates methods to append or combine 1-dimensional multimodal sample data into a single feature detector, for multimodal feature detection in accordance with illustrative embodiments of the present disclosure;

FIG. 6B illustrates methods to append or combine 2-dimensional multimodal sample data into a single feature detector, for multimodal feature detection in accordance with illustrative embodiments of the present disclosure;

FIG. 6C illustrates methods to append or combine N-dimensional multimodal sample data into a single feature detector, for multimodal feature detection in accordance with illustrative embodiments of the present disclosure;

FIG. 7A is a block diagram of a multimodal feature detection system in accordance with illustrative embodiments of the present disclosure;

FIG. 7B is a block diagram of a multimodal feature detection system with dedicated feature extractors for each sample data source prior to the machine-learned feature detection algorithm, in accordance with illustrative embodiments of the present disclosure;

FIG. 8 is a block diagram of a multimodal feature detection system with dedicated pre-processors for each sample data source prior to the machine-learned feature detection algorithm, in accordance with illustrative embodiments of the present disclosure;

FIG. 9 is a block diagram of a multimodal feature detection system with dedicated feature extractors for each sample data source prior to the machine-learned feature detection algorithm, in accordance with illustrative embodiments of the present disclosure;

FIG. 10 is a block diagram of a multimodal feature enhancer system in accordance with illustrative embodiments of the present disclosure;

FIG. 11A is a block diagram of a multimodal feature measurement system in accordance with illustrative embodiments of the present disclosure;

FIG. 11B is a block diagram of a multimodal feature detection and measurement system in accordance with illustrative embodiments of the present disclosure;

FIG. 12 is a block diagram of a multimodal feature detection algorithm in accordance with illustrative embodiments of the present disclosure;

FIG. 13A illustrates example 1-D sample data sources for a multimodal feature detection algorithm in accordance with illustrative embodiments of the present disclosure;

FIG. 13B illustrates example 1-D sample data sources for a multimodal feature detection algorithm in accordance with illustrative embodiments of the present disclosure;

FIG. 14A illustrates example 1-D sample data sources for a multimodal feature detection algorithm appended to in accordance with illustrative embodiments of the present disclosure.

FIG. 14B illustrates example 2-D sample data sources for a multimodal feature detection algorithm in accordance with illustrative embodiments of the present disclosure;

FIG. 15 illustrates example segmentation outputs overlaid on and oriented with respect to an input 2-D arterial OCT image with the example outputs being generated (e.g., predicted) using a pre-trained multimodal feature detection algorithm in accordance with illustrative embodiments of the present disclosure;

FIG. 16 illustrates example line-based segmentation outputs oriented with respect to an input 2-D arterial OCT image with the example outputs being generated (e.g., predicted) using a pre-trained multimodal feature detection algorithm in accordance with illustrative embodiments of the present disclosure;

FIG. 17 illustrates example line-based segmentation outputs oriented with respect to an input 2-D arterial OCT image with the example outputs being generated (e.g., predicted) using a pre-trained multimodal feature detection algorithm in accordance with illustrative embodiments of the present disclosure;

FIG. 18 illustrates example bounding-boxed based segmentation outputs oriented with respect to an input 2-D arterial OCT image with the example outputs being generated (e.g., predicted) using a pre-trained multimodal feature detection algorithm in accordance with illustrative embodiments of the present disclosure;

FIG. 19 illustrates an example output from a pre-trained multimodal feature enhancement algorithm in accordance with illustrative embodiments of the present disclosure;

FIG. 20 illustrates an example output from a pre-trained multimodal feature enhancement algorithm in accordance with illustrative embodiments of the present disclosure;

FIG. 21 illustrates an example output from a pre-trained multimodal feature detection and measurement algorithm in accordance with illustrative embodiments of the present disclosure;

FIG. 22 illustrates an example output from a pre-trained multimodal feature detection and measurement algorithm in accordance with illustrative embodiments of the present disclosure;

FIG. 23 illustrates an example output from a pre-trained multimodal feature detection and measurement algorithm in accordance with illustrative embodiments of the present disclosure;

FIG. 24 illustrates an example output from a pre-trained multimodal feature detection and measurement algorithm in accordance with illustrative embodiments of the present disclosure;

FIG. 25 illustrates an example display (e.g., user interface) of outputs from a pre-trained multimodal feature detection and measurement algorithm in accordance with illustrative embodiments of the present disclosure;

FIG. 26 is a block diagram of an example network environment for use in the methods and systems described herein, according to illustrative embodiments of the disclosure; and

FIG. 27 is a block diagram of an example computing device and an example mobile computing device, for use in illustrative embodiments of the disclosure.

### DETAILED DESCRIPTION OF CERTAIN EMBODIMENTS

Provided herein are, inter alia, systems and methods for using data from different characterization modalities to improve feature detection and/or extraction, feature representation, feature transformation, data (e.g., image) transformation, measurement (e.g., assessments), and combinations thereof. Thus, sample data from one characterization modality may be used to improve data (e.g., images) from another characterization modality (and vice versa). In various embodiments, machine-learning algorithms are employed to achieve such improvement. A machine-learned algorithm may receive sample data from multiple characterization modalities as input, either exclusively or in addition to other input. In some embodiments, first sample data from a first characterization modality and second sample data from a second characterization modality are provided to a machine-learned algorithm. A machine-learned algorithm may have been trained on such data. In some embodiments, an algorithm is trained on such data to generate a machine-learned algorithm. Features of interest may be detected using a machine-learned algorithm. Detection of a feature may include semantic segmentation, line based segmentation, or frame based segmentation (e.g., branch or poor quality frame).

Using methods disclosed herein, a spectroscopy modality can enhance interferometric modality (e.g., OCT) (e.g., contrast, brightness, structure, sharpening, or a combination thereof). Using methods disclosed herein, an interferometric modality (e.g., OCT) can enhance a spectroscopic modality. For example, scaling, e.g. based on distance from a lumen wall, calibration or normalization may be used to enhance spectroscopic data. Using methods disclosed herein, a detected feature (e.g., lipid) within an image (e.g., an OCT image) can be selectively enhanced, for example based on sample data from a spectroscopic characterization modality. Using methods disclosed herein, a detected feature within spectroscopy data can be selectively enhanced (e.g., to discriminate types of lipids), for example using data from an interference characterization modality, such as OCT.

In some embodiments, a method and/or system disclosed herein employs a machine-learned algorithm such as a neural network, random decision forest, support vector machine or other machine-learned model. A machine-learned algorithm may employ (e.g., perform) segmentation and/or classification, for example to detect one or more features of interest, determine one or more measurements (e.g., of one or more feature representations of one or more features of interest), transform (e.g., enhance) a feature representation of one or more features of interest, or a combination thereof. A machine-learned algorithm may perform multiple such functions sequentially or simultaneously. A machine-learned algorithm may use a single stage or multiple stages, for example different stages may be used to perform different such functions (e.g., based on different inputs), such as a first stage that performs feature extraction/detection and a second stage that determines one or more measurements. A machine-learned algorithm may be or include a feature detector and/or feature extractor.

In some embodiments, a machine-learned algorithm includes (e.g., is) a classifier which has been trained using supervised learning or semi-supervised learning and using a training data set. The training data set for such a classifier algorithm generally includes a plurality of training examples where each training example is test data of a sample and a ground truth value of the class of the sample. Training examples may be obtained empirically and/or be synthetic training examples computed using a software simulation. In some embodiments, empirical training examples are generated from one or more catheterization procedures that image one or more portions of one or more subjects (e.g., human(s)), for example from a population of subjects that exhibit a range of physiologies. A range of different samples may be obtained, such as from human subjects from different demographic groups so as to avoid any unintentional bias in performance of the resulting machine learning model. For each sample used to form a training data example, a class of a sample (e.g., healthy tissue, plaque morphology, and/or calcium morphology) may be known from medical records of the human subject from which the sample is derived. A machine-learned algorithm may then be trained using any suitable training algorithm and an objective function which takes into account differences between predictions of the algorithm and the ground truth classes of the training examples. In some embodiments where the machine learning model is a neural network such as a multi-layer perceptron or other type of neural network, the neural network may be trained using backpropagation. A machine-learned algorithm may then be trained using the available training data items or until little change in parameters of the machine learning model are observed. Once a machine-learned algorithm has been trained it may then be deployed at any suitable computing device such as a computing device included in an imaging catheter system, a hospital desktop computer, or a web server in the cloud, for example. A deployed machine-learned algorithm may receive a test data item from a sample not previously used to train the algorithm, for example in order to detect one or more features of interest, to determine one or more measurements, to transform a representation of one or more features of interest, or a combination thereof. In some embodiments, a machine-learned algorithm processes the test data item to compute a prediction indicating which class of a plurality of classes the test data falls into. In some cases, a machine-learned algorithm provides a confidence value indicating uncertainty associated with a prediction. In some embodiments, a machine-learned algorithm performs segmentation in addition to classification, such an algorithm may be trained to perform segmentation using a similar approach as with classification.

In general, any suitable training approach may be used to form a machine-learned algorithm. In some embodiments, a predicate system is used to train a multimodal system as disclosed herein. In some embodiments, training is based on, at least, manually annotated data. For example, an expert, such as an appropriate physician (e.g., cardiologist), may annotate data (e.g., images) that is then input as training data to form a machine-learned algorithm. For example, a cardiologist may annotate plaque locations, sizes, or other parameters within a larger data set. Such annotations may be made to data oriented in a polar or Cartesian coordinate system, for example. In some embodiments, output (e.g., co-registered output) from another device may be used to train an algorithm. In some embodiments, registered ground-truth histology data is used to train an algorithm. Any combination of these (manual annotation, co-registered output from another device, registered ground-truth) may also be used to train an algorithm into a machine-learned algorithm. Any one or combination of these approaches (manual annotation, co-registered output from another device, registered ground-truth) may be used to generate training labels that are then associated with training data. Training labels may be derived from data from one characterization modality while training data with which the labels are used is data from a different characterization modality. For example, training labels derived from data from a spectroscopic modality may be used with training data from an interferometric modality (e.g., using co-registered data sets from the two modalities to generate the labelled training data).

In some embodiments, an algorithm is or has been trained to detect one or more objects (e.g., plaque). In some embodiments, such detection is based on only a first characterization modality [e.g., an interferometric modality (e.g., OCT)]. The training of such an algorithm may be performed using training labels derived from data from a second characterization modality [e.g., a spectroscopic modality (e.g., NIRS)]. In some embodiments, the use of co-registered multimodal data enables such algorithm training, for example where training data is derived from one modality and training labels for the training data are derived from another modality. Accordingly, in some embodiments, detection and/or transformation (e.g., enhancement) of one or more features of interest and/or measurement thereof may use data from only one characterization modality as an input to a machine-learned algorithm that has been trained using data labeled based on another characterization modality. One example of such an algorithm is one where objects (e.g., lipid core plaques) are detected and/or transformed (e.g., enhanced) using interferometric modality (e.g., OCT) data only where spectroscopic (e.g., NIRS) data was nonetheless used for training labels. In some embodiments, training labels are also derived from a first characterization modality corresponding to the modality used to generate the training data (e.g., training labels are derived from two modalities while training data is derived from only one of those two modalities).

Thus, in some embodiments, improved feature detection (e.g., segmentation and/or classification) and/or transformation (e.g., enhancement) and/or measurement thereof can be realized from sample data from only one characterization modality by using a machine-learned algorithm that has been trained based on information derived from more than one characterization modality. Benefit(s) of a second characterization modality can be realized where only sample data from a first characterization modality are available using some such machine-learned algorithms. For example, spectroscopic modalities may be better suited to characterize composition of certain features of interest as compared to interferometric modalities and therefore feature detection and/or transformation and/or measurement thereof may be improved using training data from an interferometric modality that has been labelled based on data from a spectroscopic modality. Such approaches may be useful, for example, where sample data is collected with a device, such as a catheter, that is a single modality device or where multimodal data are poorly co-registered or unable to be co-registered for some reason.

In some embodiments, a machine-learned algorithm outputs a feature representation [e.g., a transformed (e.g., enhanced) feature representation] and/or one or more measurements. In some embodiments, outputting comprising displaying the feature representation, for example software may be programmed to automatically display data output from a machine-learned algorithm for viewing by a user. In some embodiments, outputting comprises saving (e.g., storing) and/or sending the feature representation to another computing device (e.g., a hospital PACS). One or more measurements may be displayed near (e.g., adjacent to) and/or overlaid onto one or more images. An image may be a combined representation of multiple characterization modalities, for example corresponding to a combination of an interference modality and a spectroscopy modality. As disclosed herein, a machine-learned algorithm may be used to transform a representation of a feature of interest corresponding to one modality using data from another modality (e.g., and vice versa). A feature representation generated using a machine-learned algorithm may be combined with image data that has not been modified using a machine-learned algorithm and displayed to a user. For example, a machine-learned algorithm may be used to detect a feature of interest and generate a feature representation, optionally a transformed feature representation, and output that representation (e.g., transformed representation) such that a composite image that includes the feature representation and any unaffected surrounding data in a single image. For example, a plaque and/or calcium morphology may be generated as a feature representation output from a machine-learned algorithm that is then used in an image that includes a representation of surrounding tissue derived from original sample data that is unaltered by a machine-learned algorithm (e.g., not input into the machine-learned algorithm). In some embodiments, an entire (e.g., displayed) image is generated as output from a machine-learned algorithm, for example where only one or more (e.g., detected) features of interest are portrayed as transformed representations. In some embodiments, a feature representation output is registered to first sample data, second sample data, or both first sample data and second sample data. For example, first sample data may be, or may otherwise be useable to derive, an OCT image and a feature representation output of a machine-learned algorithm may be registered to the first sample data.

A machine-learned algorithm may employ, for example, a regression-based model (e.g., a logistic regression model), a regularization-based model (e.g., an elastic net model or a ridge regression model), an instance-based model (e.g., a support vector machine or a k-nearest neighbor model), a Bayesian-based model (e.g., a naive-based model or a Gaussian naive-based model), a clustering-based model (e.g., an expectation maximization model), an ensemble-based model (e.g., an adaptive boosting model, a random forest model, a bootstrap-aggregation model, or a gradient boosting machine model), or a neural-network-based model (e.g., a convolutional neural network, a recurrent neural network, autoencoder, a back propagation network, or a stochastic gradient descent network). In embodiments, a machine learning model is trained using supervised learning algorithms, unsupervised learning algorithms, semi-supervised learning algorithms (e.g., partial supervision), weak supervision, transfer, multi-task learning, or any combination thereof. In some embodiments, a machine-learned algorithm employs a model that comprises parameters (e.g., weights) that are tuned during training of the model. For example, the parameters may be adjusted to minimize a loss function, thereby improving the predictive capacity of the machine-learned algorithm.

In some embodiments, a machine-learned algorithm uses multimodal data (e.g., including interferometric data and spectroscopic (e.g., diffuse spectroscopy) data, e.g. appended to each other) to detect one or more features of interest in sample data (e.g., one or more images). In general, a feature of interest may be any feature that it is of interest to a particular user for characterization. In some embodiments, systems and methods disclosed herein are used to characterize subjects (e.g., humans). For example, a system may be an imaging catheter system, such as a system for intravascular imaging. In some embodiments, a feature of interest may be an internal or external structure (e.g., physiological structure) of a subject. A system disclosed herein (e.g., an imaging catheter system) may be used to image, for example, one or more blood vessels (e.g., arteries) and/or one or more organs (e.g., eye(s)). A feature of interest may be a structure in and/or on such a vessel or organ. The following are non-limiting examples of objects that can be detected (e.g., in one or more images) (e.g., segmented and/or classified) using methods disclosed herein (e.g., using interferometric data and spectroscopic data). In some embodiments, any one or more of these objects may be a feature of interest. One or more artery wall structures may be detected. For example a lumen, an external elastic membrane (EEM), external elastic lamina (EEL), an intima, media, adventitia, side branches, calcium, lipid, lipid subtypes, calcium subtypes, collagen, cholesterol, or a combination thereof may be a feature of interest (e.g., that is detected and/or a representation of which is measured). Cap thickness over a lipid pool/necrotic core may be detected and/or measured. Artery pathologies may be one or more features of interest (e.g., detected), for example pathological intimal thickening, intimal xanthoma, early and late fibroatheroma, thin cap fibroatheroma, one or more fibro-lipidic plaques, or one or more fibro-calcific plaques. Foreign objects may be one or more features of interest (e.g., detected), for example, a catheter, probe lens, probe reflector, probe (e.g., catheter) sheath, guidewire, stent(s), or a combination thereof. Curvature of a vessel may be a feature of interest.

In some embodiments, a machine-learned algorithm outputs a feature representation of a feature of interest. An algorithm may output different feature representations for different features of interest or a common representation that includes multiple features of interest. A feature representation may be generated from a feature of interest detected by an algorithm. In general, a feature representation is data. Data that define a feature representation may be sufficient to display as an image, for example on a display included in a multimodal characterization system. A feature representation may be simply saved (e.g., stored) and/or sent, for example as opposed to being displayed. Data that define a feature representation may define only a portion of an image, for example an image that represents a portion of a subject, such as a blood vessel. For example, data that define a feature representation may be integrated with (e.g., appended to) other data to form a complete image of the portion of the subject. A feature representation may include a representation of a feature of interest along with other information (e.g., sample data) that does not correspond to the feature of interest (e.g., other structure(s) surrounding the feature of interest in or on a subject). A feature representation may correspond to one or more characterization modalities, for example be representative of data from an interference modality and a spectroscopic modality. A feature representation may be generated using a machine-learned algorithm based on sample data from multiple characterization modalities. A machine-learned algorithm may output one or more transformed (e.g., enhanced) feature representations, for example of one or more features of interest. In some embodiments, multiple feature representations of a single feature of interest are output, for example to provide a user (e.g., physician) with a more holistic assessment of the feature of interest.

One or more measurements may be determined (e.g., automatically) (e.g., using a machine-learned algorithm) based on feature representation data output from a machine-learned algorithm. For example, a feature of interest may be a lipid core; a machine-learned algorithm may detect the presence of that lipid core and generate data that define a feature representation of that lipid core; and one or more measurements, for example core area, volume, and/or thickness, may be determined using the feature representation data. Generating data that defines a feature representation may include processing first sample data from a first characterization modality, second sample data from a second characterization modality, or both such first sample data and such second sample data. Generating data that defines a feature representation may include selectively segmenting/extracting first sample data from a first characterization modality, second sample data from a second characterization modality, or both such first sample data and such second sample data that is identified as corresponding to a feature of interest.

An image, or portion thereof may be transformed (e.g., enhanced) using a machine-learned algorithm. For example, a feature representation included in an image may be transformed (e.g., enhanced) using a machine-learned algorithm. Enhancement of image data may include one or more of: processing, pre-processing, improving (e.g., resolution), contrast, scale, and ratio. A transformation of at least a portion of an image (e.g., feature representation therein) (e.g., whole image) may result in a new imaging space or color scheme. In some embodiments, an image (e.g., an OCT image) (e.g., derived from sample data from an interference modality) is transformed to another image space (e.g., white light image, false histology staining) based on spectroscopy data (e.g., from visible diffuse reflectance spectroscopy, or infrared spectroscopy or auto-fluorescence). Using systems and methods disclosed herein, a specular versus diffuse reflection ratio can be measured using one data source (from a first characterization modality) and that measurement data can be used to improve or enhance attenuation correction for a second data source (from a second characterization modality). For example, spectroscopy could be used to evaluate such a ratio, while the ratio could be used to provide a more accurate attenuation correction for interferometric (e.g., OCT) data.

The following are non-limiting examples of measurements that can be made (e.g., in one or more images) using methods disclosed herein (e.g., using interferometric data and spectroscopic data): stent malapposition, stent length, stent location planning, EEM measurements, EEL measurements, lumen area, plaque burden, lipid measurement(s) (e.g., arc, lipid core burden, max lipid core burden over a distance, thickness, extent, area, volume, ratio lipid to other), calcium measurement(s) (e.g., arc, thickness, extent, area, volume, ratio lipid to other), necrotic core cap thickness, plaque vulnerability, lipid core burden, and combinations thereof. In some embodiments, a machine-learned algorithm performs a measurement.

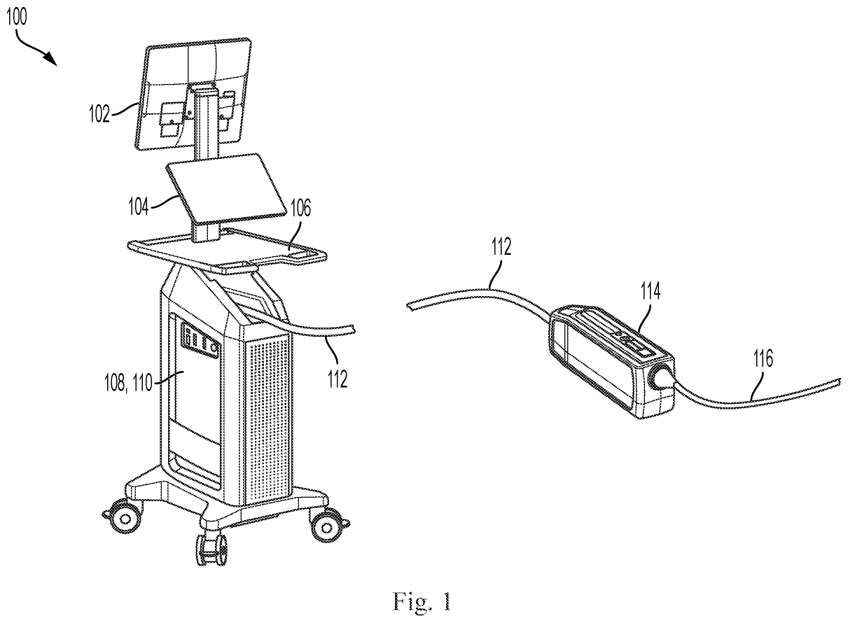

FIG. 1 illustrates a block diagram of a multimodal characterization (e.g., feature detection) system in accordance with illustrative embodiments of the present disclosure. System 100 includes physician display 102, a technician display 104, a tray 106, computing device 108 that includes a processor, a memory, and one or more machine-learned algorithms as disclosed herein, stationary unit 110, fiber 112, rotary unit 114, and probe 116. System 100 as illustrated is an imaging catheter system; probe 116 is an imaging catheter, for example a cardiac catheter. In some embodiments, a multimodal characterization system includes a probe but is not an imaging catheter. Not all of fiber 112 is shown (as indicated by the slashes), to indicate that fiber 112 may be, in general, of any suitable length. Stationary unit 110, rotary unit 114, fiber 112, and probe 116 are together operable to perform (e.g., simultaneously) multiple characterization modalities (e.g., during a pullback of probe 116). For example, stationary unit 110 and/or rotary unit 114 may include one or more detectors for such purpose.

In some embodiments, at least one characterization modality in a multimodality system is an interference technique (e.g., OCT). In some embodiments, at least one characterization modality in a multimodality system is a spectroscopy technique (e.g., NIRS). In general, a system may include one or more characterization modality subsystems (e.g., two different characterization modality subsystems). A characterization modality subsystem may be an interferometric modality (e.g., OCT) subsystem or a spectroscopy modality (e.g., DRS, such as NIRS) subsystem. Different characterization modality subsystems may share components, such as, for example, optics and/or fibers. Different characterization modality subsystems may have at least some different components, such as detectors and/or light sources.

Examples of multimodality characterization systems that may be used as, adapted into, or modified to be a multimodality system as disclosed herein are disclosed in International (PCT) Patent Application No. PCT/US22/40409, filed on Aug. 16, 2022, the disclosure of which is hereby incorporated by reference herein in its entirety. Examples of probes that may be used in a multimodality characterization system as disclosed herein are disclosed in International (PCT) Patent Application No. PCT/US22/50460, filed on Nov. 18, 2022, the disclosure of which is hereby incorporated by reference herein in its entirety.

Output (e.g., one or more feature representations of one or more detected features and/or one or more measurements of the one or more detected features) from the machine-learned algorithm(s) may be displayed on display 102. In some embodiments, display 102 is used by a user (e.g., physician) to control system 100 but output from the machine-learned algorithm(s) is not displayed but is saved and/or sent elsewhere (e.g., to a hospital PACS). For example, a user may be able to choose whether to display output or simply save and/or send the output for future display elsewhere. Output may be both displayed on display 102 and saved and/or sent elsewhere. System 100 may be located in or near a procedure room, such as a catheterization lab.

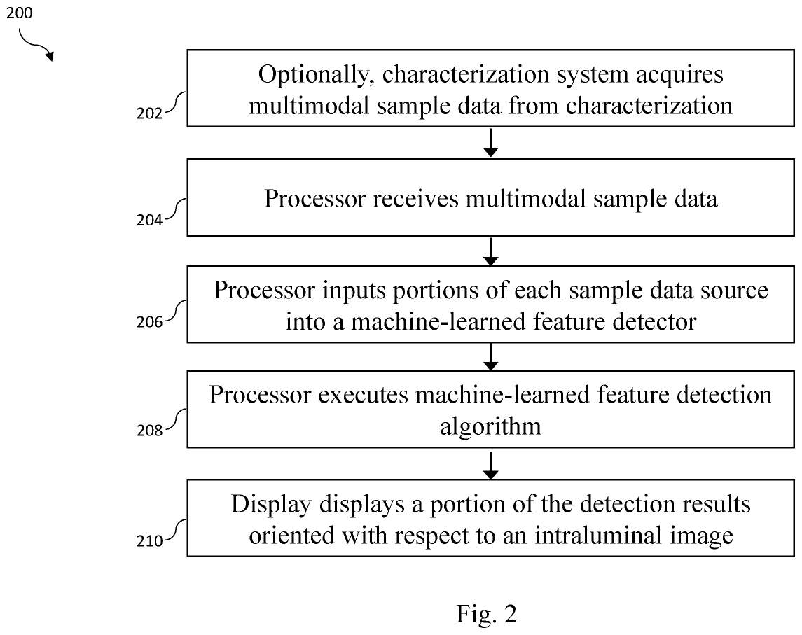

Machine-learned algorithms as disclosed herein may perform one or more functions, including, for example, feature detection, feature transformation (e.g., enhancement) (e.g., of detected features), feature measurement (e.g., of detected features), or a combination thereof. FIGS. 2-5 illustrate example methods that utilize a system that includes a machine-learned algorithm. A system may include one or more characterization modality subsystems that are used to generate sample data and/or may receive sample data from elsewhere (e.g., sent by one or more separate systems that is/are used for data acquisition).

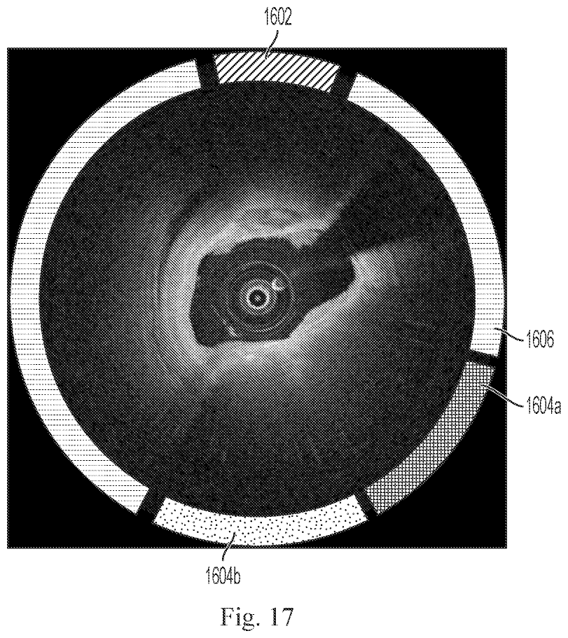

FIG. 2 is a flowchart of a method 200 of using a multimodal feature detection system in accordance with illustrative embodiments of the present disclosure. Method 200 starts with step 202. In optional step 202, a characterization system acquires multimodal data from characterization modality subsystems. Multimodal data generally includes at least first sample data from a first characterization modality and second sample data from a second characterization modality. In step 204, a processor receives the multimodal sample data, including the first sample data and the second sample data. In step 206, the first sample data and the second sample data are provided to a machine-learned algorithm, which includes the processor inputting portions of the sample data into the machine-learned algorithm, which in this case is a machine-learned feature detector. In general, when sample data is provided to a machine-learned algorithm, not necessarily all of the data are input into the machine-learned algorithm. In some embodiments, sample data provided to a machine-learned algorithm are first processed (e.g., run through a feature extractor) and/or pre-processed (e.g., filtered) before actually being input into the machine-learned algorithm. Referring back to FIG. 2, in step 208, the processor executes the machine-learned feature detector based on the input, which may include other data in addition to the first sample data and second sample data. The machine-learned feature detector outputs, for example, one or more feature representations of one or more features of interest detected by the algorithm. In step 210, a display displays at least a portion of the detection results, which may be oriented with respect to an intraluminal image. For example, feature representation(s) of detected feature(s) may be overlaid over an image derived from the original first sample data or the original second sample data. FIGS. 15-25, described further subsequently, give examples of such overlaid representations.

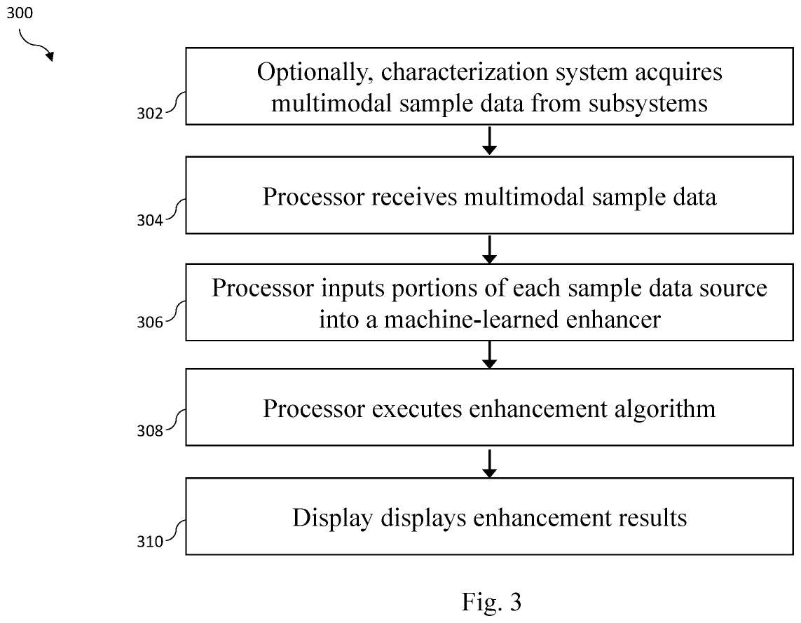

FIG. 3 is a flowchart of a method 300 of using a multimodal feature enhancer system in accordance with illustrative embodiments of the present disclosure. In optional step 302, a characterization system acquires multimodal data from characterization modality subsystems. Multimodal data generally includes at least first sample data from a first characterization modality and second sample data from a second characterization modality. In step 304, a processor receives the multimodal sample data, including the first sample data and the second sample data. In step 306, the first sample data and the second sample data are provided to a machine-learned algorithm, which includes the processor inputting portions of the sample data into the machine-learned algorithm, which in this case is a machine-learned feature transformer that is a machine-learned feature enhancer. In step 308, the processor executes the machine-learned enhancement algorithm based on the input, which may include other data in addition to the first sample data and second sample data. The machine-learned feature detector outputs, for example, one or more transformed, in this case enhanced, feature representations of one or more features of interest detected by the algorithm. The machine-learned algorithm may first detect feature(s) of interest before, or while simultaneously, enhancing them. In step 210, a display displays at least a portion of the enhanced results, which may be oriented with respect to an intraluminal image. For example, enhanced feature representation(s) of detected feature(s) may be overlaid over an image derived from the original first sample data or the original second sample data.

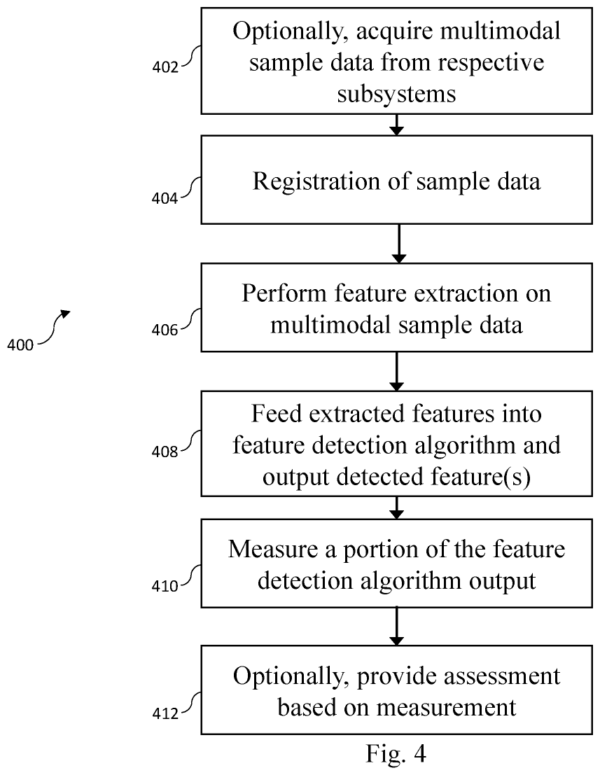

FIG. 4 is a flowchart of a method 400 of using a multimodal feature detection and measurement system in accordance with illustrative embodiments of the present disclosure. In optional step 402, a characterization system acquires multimodal data from characterization modality subsystems. Multimodal data generally includes at least first sample data from a first characterization modality and second sample data from a second characterization modality. In step 404, the first sample data and the second sample data are (co-)registered. In some embodiments, data are automatically co-registered due to how they are acquired. For example, some characterization systems, such as certain multimodal intravascular catheter systems, acquire data that are sufficiently co-registered based on simultaneous data collection for multiple modalities. In step 406, processing, in this case feature extraction, is performed on the first sample data and the second sample data prior to input into a machine-learned algorithm. Feature extraction at this step may involve use of a machine-learned algorithm or may involve other known feature extraction techniques. In step 408, the first sample data and the second sample data are provided to the machine-learned algorithm, a feature detection and measurement algorithm, and one or more detected feature(s) are output, for example in the form of one or more feature representations. In particular, the extracted features from step 406 are fed, as input, into the algorithm. In step 410, one or more measurements are determined with at least a portion of the feature representation output. Measurements may be determined with a machine-learned algorithm, such as the machine-learned feature detector and measurement algorithm. In some embodiments, an assessment is provided based on measurement(s) made, for example in optional step 412. The assessment may itself be a measurement in certain embodiments, such as, for example, when the assessment is an index, like a plaque burden index. An assessment may be made in any suitable fashion, for example automatically by a processor and/or using a machine-learned algorithm (e.g., that uses one or more measurements, first sample data from a first characterization modality, second sample data from a second characterization modality, or some combination thereof).

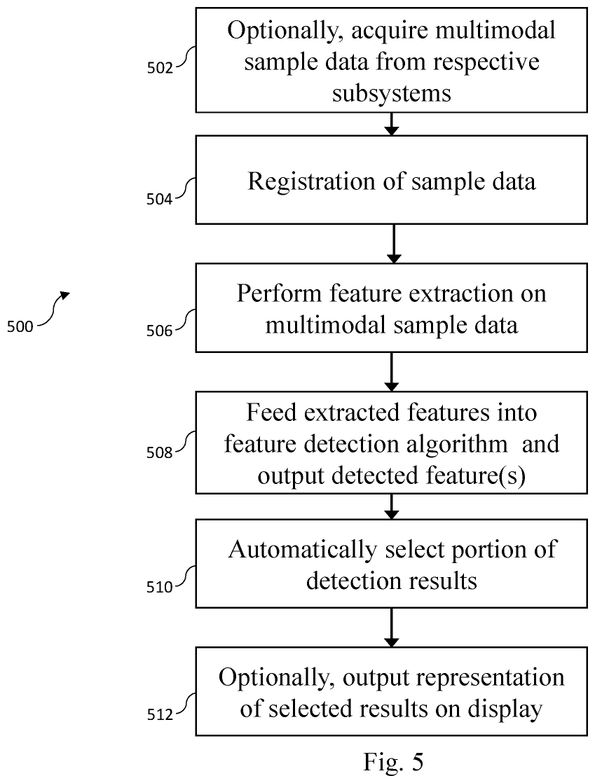

FIG. 5 is a flowchart of a method 500 of using a multimodal feature detection system in accordance with illustrative embodiments of the present disclosure. In optional step 502, a characterization system acquires multimodal data from characterization modality subsystems. Multimodal data generally includes at least first sample data from a first characterization modality and second sample data from a second characterization modality. In step 504, the first sample data and the second sample data are (co-)registered. In step 506, processing, in this case feature extraction, is performed on the first sample data and the second sample data prior to input into a machine-learned algorithm. Feature extraction at this step may involve use of a machine-learned algorithm or may involve other known feature extraction techniques. In step 508, the first sample data and the second sample data are provided to the machine-learned algorithm, a feature detection and measurement algorithm, and one or more detected feature(s) are output, for example in the form of one or more feature representations. In particular, the extracted features from step 506 are fed, as input, into the algorithm. In step 510, at least a portion of the detection results are automatically selected (e.g., corresponding to one or more features of interest). The output of the detected feature(s) in step 508 may be in the form of one or more feature representations and/or the automatic selection in step 510 may include selecting one or more feature representations (e.g., including generating the representation(s) from certain detected feature(s)). In optional step 512, feature representation(s) of selected results are output on a display.

In some embodiments, at least two data sets are provided to a machine-learned algorithm. For example, at least first sample data from a first characterization modality and second sample data from a second characterization modality are provided to a machine-learned algorithm. In general, a set of sample data can be in any suitable form in any suitable dimension, for example a vector, which may be a scalar value (i.e., a 1×1 dimensioned vector). Data sets may be appended to each other to act as input to a machine-learned feature detector. For example, a machine-learned algorithm may accept a single input that includes data from multiple characterization modalities, instead of two separate inputs. In some embodiments, input to a machine-learned algorithm has a reduced dimensionality input as compared initial first sample data and/or second sample data; thus, processing may be required to prepare first sample data and second sample data for input to a machine-learned algorithm. In some embodiments, appending includes merging two or more data sources into one in any dimension. Sample data may include data from one or more characterization subsystems, such as an interferometric modality subsystem (e.g., OCT) and/or spectroscopic modality subsystem (e.g., diffuse spectroscopy, e.g. NIRS). Sample data may include interferometric data and/or spectroscopic data.

First sample data from a first characterization modality and second sample data from a second characterization modality may be (co-)registered, for example either automatically due to how they are acquired or by post-acquisition processing. For example, two characterization modalities may be performed simultaneously, as is the case for certain systems, such as multimodal imaging catheter systems (e.g., intraluminal catheter systems). Nonetheless, co-registered sample data from different modalities does not mean that the sample data necessarily correspond to instantaneously contemporaneous acquisition and/or that the sample data necessarily correspond to coextensive sample volumes.

In some embodiments, first sample data are generated from a first characterization modality detected at a first region having a first volume (e.g., a first tissue volume within a bodily lumen) and the second sample data are generated from a second characterization modality detected at a second region having a second volume (e.g., a second tissue volume within a bodily lumen). The first sample data and the second sample data may then be provided to a machine-learned algorithm. This first region and second region may be only a portion of the overall sample volume that is characterized. For example, in an imaging catheter system, sample data collection generally occurs at a high frequency (e.g., >1 kHz) in order to fully image a sample [e.g., an intraluminal volume (e.g., an artery)] and the first region and the second region may only refer to a single data collection instance or may refer to multiple ones (e.g., all) of the instances. In some embodiments, the first region and the second region do not completely overlap. In some embodiments, a characterization volume (e.g., an intraluminal characterization volume) of each of a plurality of characterization modalities does not completely overlap and sample data corresponding to each of the characterization volumes is provided to a machine-learned algorithm. Different sample regions may result from, for example, different optical properties of light used (e.g., provided as illumination light and/or detected) for different characterization modalities with respect to a sample being characterized. For example, OCT and NIRS applied to the same sample using a common probe will generally detect light from different sample volumes due to differences in optical characteristics relevant to the two modalities. In some embodiments, a machine-learned algorithm operates during data acquisition.

While the preceding paragraph discussed ways in which first sample data and second sample data may correspond to not entirely spatially coextensive regions in a sample, first sample data and second sample data may, alternatively or additionally, correspond to not entirely coextensive temporal periods. For example, in some embodiments, first sample data are generated from detection with a first characterization modality at a time t₁ and second sample data are generated from detection with a second characterization modality at time t₂ where t₂−t₂>0. In some embodiments, t₂−t₁<5 ms (e.g., <3 ms, <2 ms, or <1 ms).

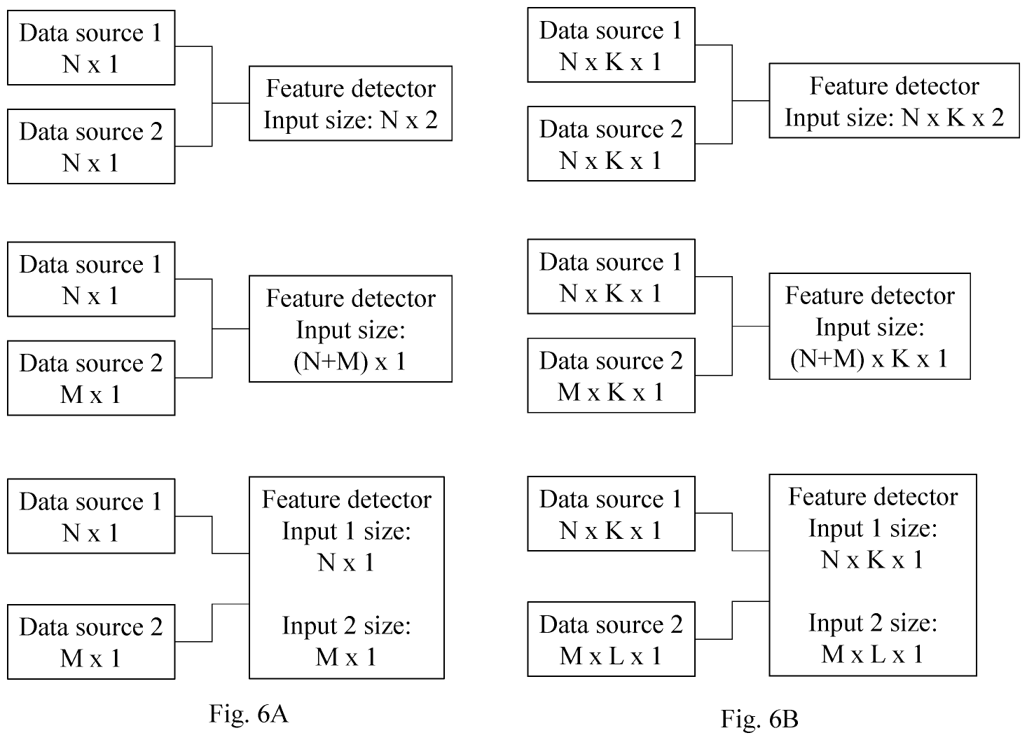

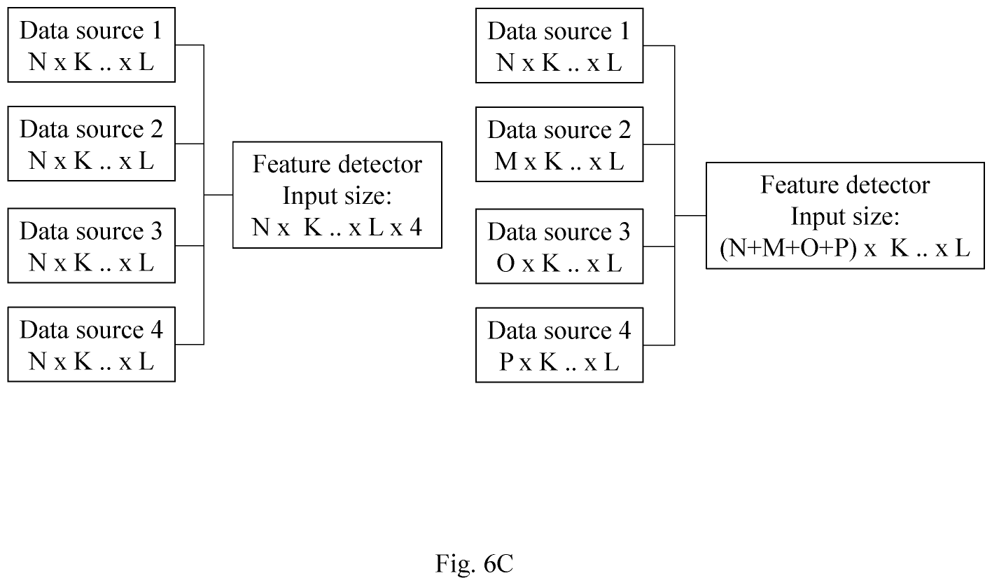

FIGS. 6A-6C illustrate certain exemplary data sets, represented by their dimensionality, and inputs to machine-learned algorithms (referred to in the figures as “feature detectors”). FIG. 6A illustrates methods to append or combine 1-dimensional multimodal sample data (including first sample data from a first characterization modality and second sample data from a second characterization modality) together into a single feature detector, for multimodal feature detection in accordance with illustrative embodiments of the present disclosure. FIG. 6B illustrates methods to append or combine 2-dimensional multimodal sample data (including first sample data from a first characterization modality and second sample data from a second characterization modality) into a single feature detector, for multimodal feature detection in accordance with illustrative embodiments of the present disclosure. FIG. 6C illustrates methods to append or combine N-dimensional multimodal sample data (including first sample data from a first characterization modality and second sample data from a second characterization modality) into a single feature detector, for multimodal feature detection in accordance with illustrative embodiments of the present disclosure.

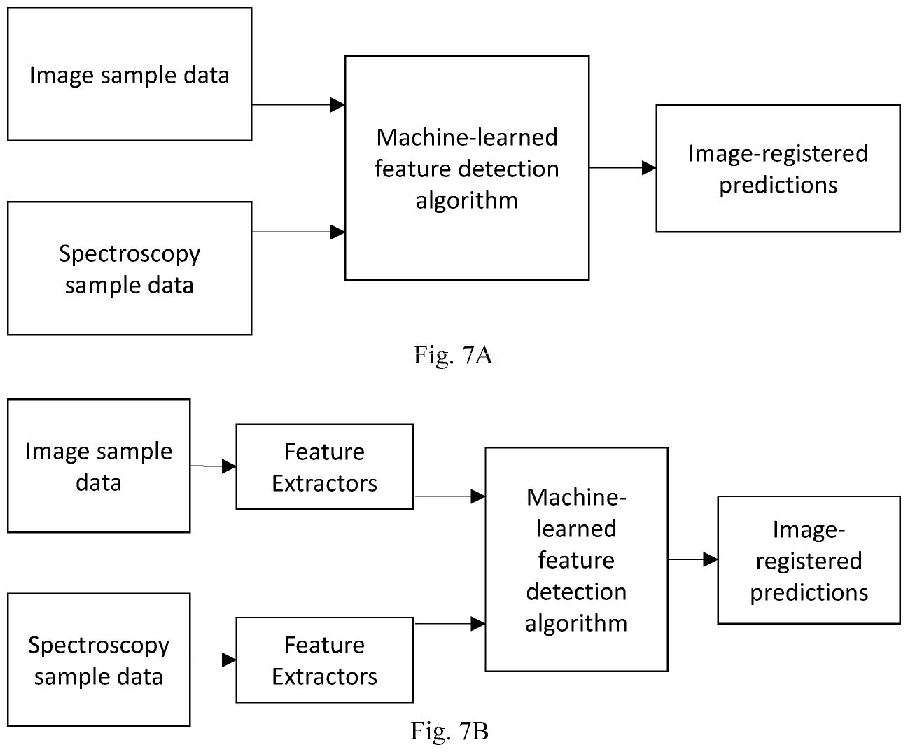

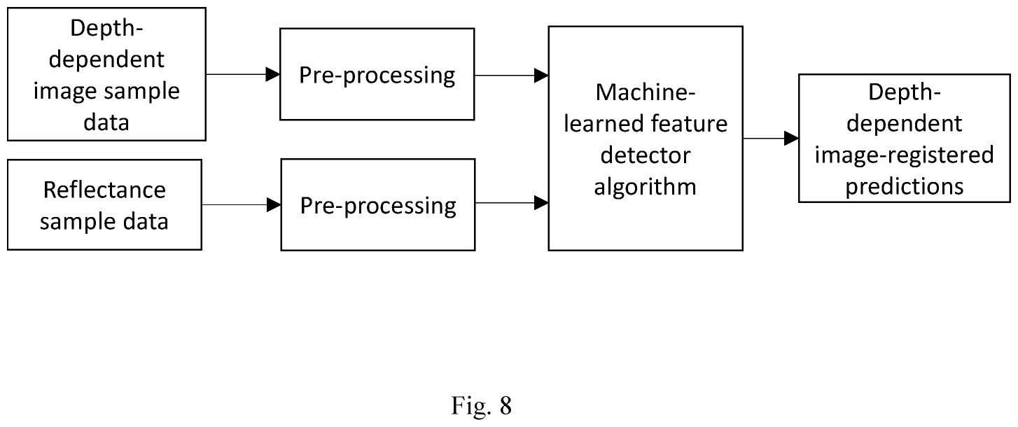

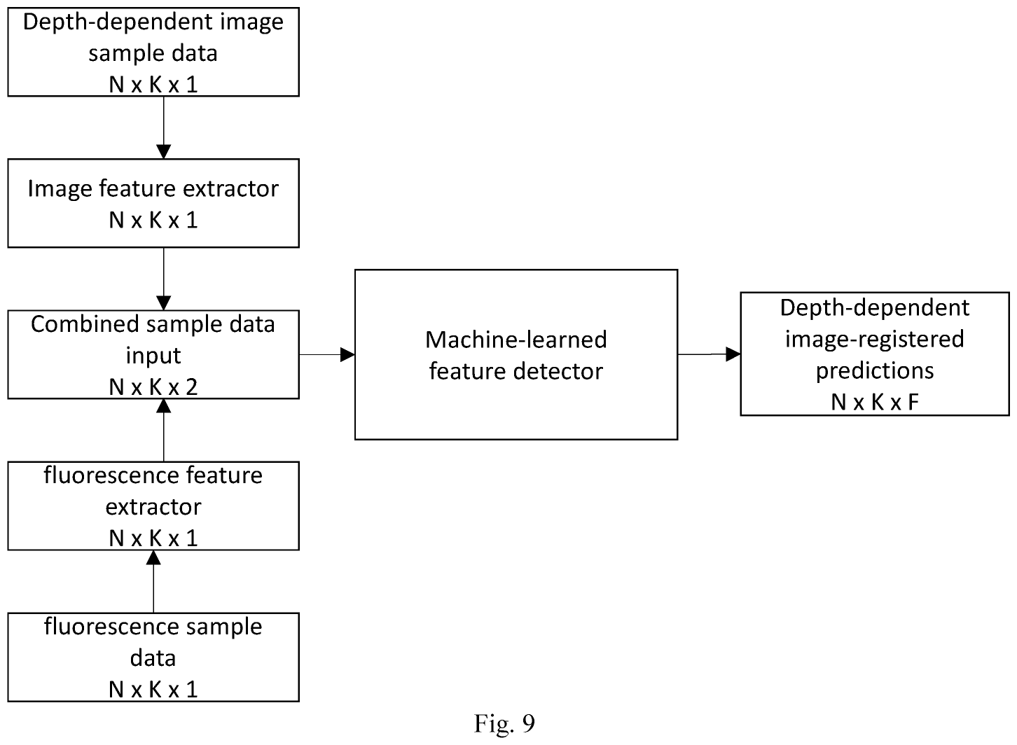

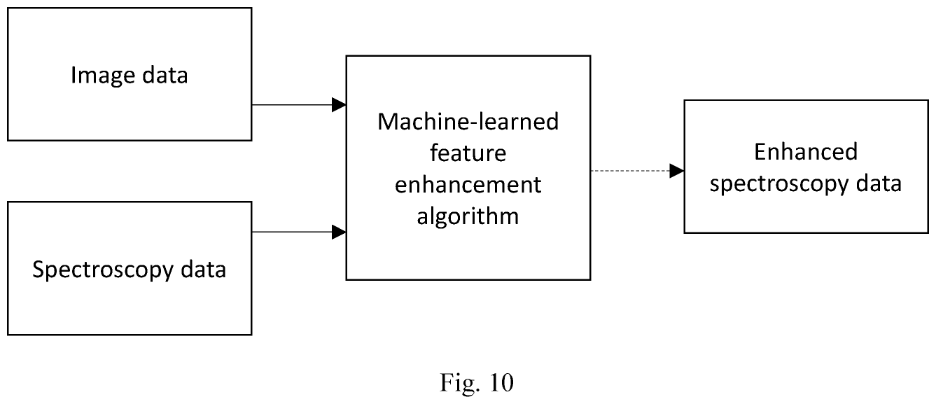

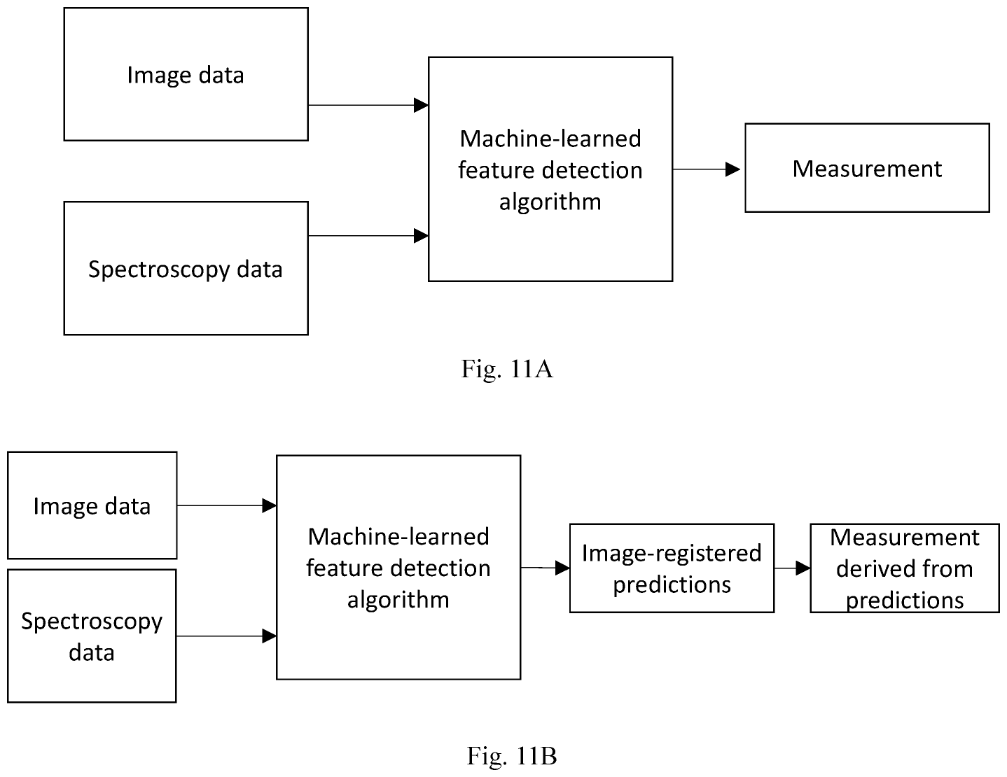

FIGS. 7A-11B are block diagrams of illustrative multimodal characterization systems that include a machine-learned algorithm, where the machine-learned algorithm perform feature detection, feature representation transformation (e.g., enhancement), measurement (e.g., on a feature representation for a feature of interest), or a combination thereof. FIG. 7A is a block diagram of an illustrative multimodal feature detection system in accordance with illustrative embodiments of the present disclosure. In this example, sample data inputs include image data and spectroscopy data while the outputs of the machine-learned algorithm are image-registered predictions. FIG. 7B is a block diagram of an illustrative multimodal feature detection system with dedicated feature extractors for each sample data source prior to the machine-learned feature detection algorithm, in accordance with illustrative embodiments of the present disclosure. In this example, sample data inputs include image data (e.g., OCT data) and spectroscopy data while the outputs of the machine-learned algorithm are image-registered predictions. The feature extractors process the image sample data and the spectroscopy sample data prior to them being provided to the machine-learned algorithm. FIG. 8 is a block diagram of an illustrative multimodal feature detection system with dedicated pre-processors for each sample data source prior to the machine-learned feature detection algorithm. In this example, sample data inputs include depth-dependent image data (e.g., OCT data) and reflectance measurement data while the outputs of the machine-learned algorithm are image-registered predictions. FIG. 9 is a block diagram of a multimodal feature detection system with dedicated feature extractors for each sample data source prior to the machine-learned feature detection algorithm. In this example, sample data inputs include depth-dependent image data (e.g., OCT data) and fluorescence data while the outputs of the machine-learned algorithm are image-registered predictions. FIG. 10 is a block diagram of a multimodal feature transformation (e.g., enhancer) system in accordance with illustrative embodiments of the present disclosure. In this example, sample data inputs include image data (e.g., OCT data) and spectroscopy data while the output of the machine-learned algorithm is enhanced spectroscopy data. In some embodiments, the output of the machine-learned algorithm is enhanced image data (e.g., an enhanced OCT image). FIG. 11A is a block diagram of a multimodal feature measurement system in accordance with illustrative embodiments of the present disclosure. In this example, sample data inputs include image data (e.g., OCT data) and spectroscopy data while the outputs of the machine-learned algorithm are feature measurements. FIG. 11B is a block diagram of a multimodal feature detection and measurement system in accordance with illustrative embodiments of the present disclosure. In this example, sample data inputs include image data (e.g., OCT data) and spectroscopy data while the outputs of the machine-learned algorithm are detected features and subsequent measurements on the detected features.

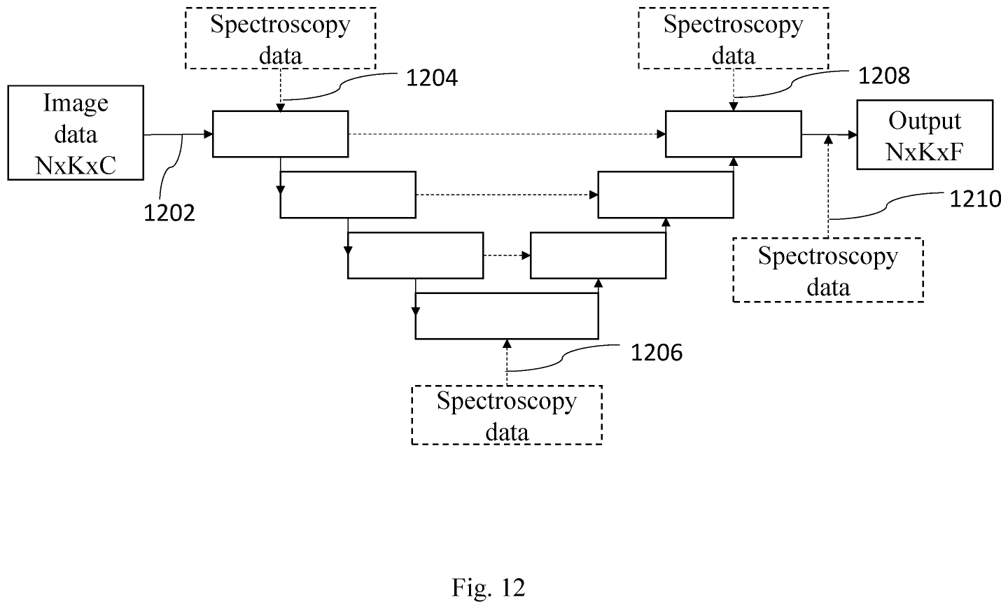

FIG. 12 is a block diagram of a multimodal feature detection algorithm in accordance with illustrative embodiments of the present disclosure. In this example, sample data inputs include image data (e.g., OCT data) and spectroscopy data while the outputs of the machine-learned algorithm are detected features (e.g., in the form of feature representations). As illustrated, the spectroscopy sample data may be input at different layers 1202 of a multi-stage neural network, including: the input layer 1204 (e.g., a convolutional layer), a mid-stage layer 1206 (e.g., a latent/encoded/down-sampled layer), an end-stage layer 1208 (e.g. an up-sampled layer) or even in the loss function 1210, or in a combination thereof. This example is not limiting. For example, spectroscopy sample data may be input in an input layer while image data (e.g., OCT data) are input in the input layer, a mid-stage layer (e.g., a latent/encoded/down-sampled layer), an end-stage layer (e.g., an up-sampled layer), in a loss function, or a combination thereof. As another example, both image data (e.g., OCT data) and spectroscopy data may be input at one or more of any of different stages of a multi-stage machine-learned algorithm. In general, data from one or more characterization modalities may be input at one or more of any of different stages of a multi-stage machine-learned algorithm.

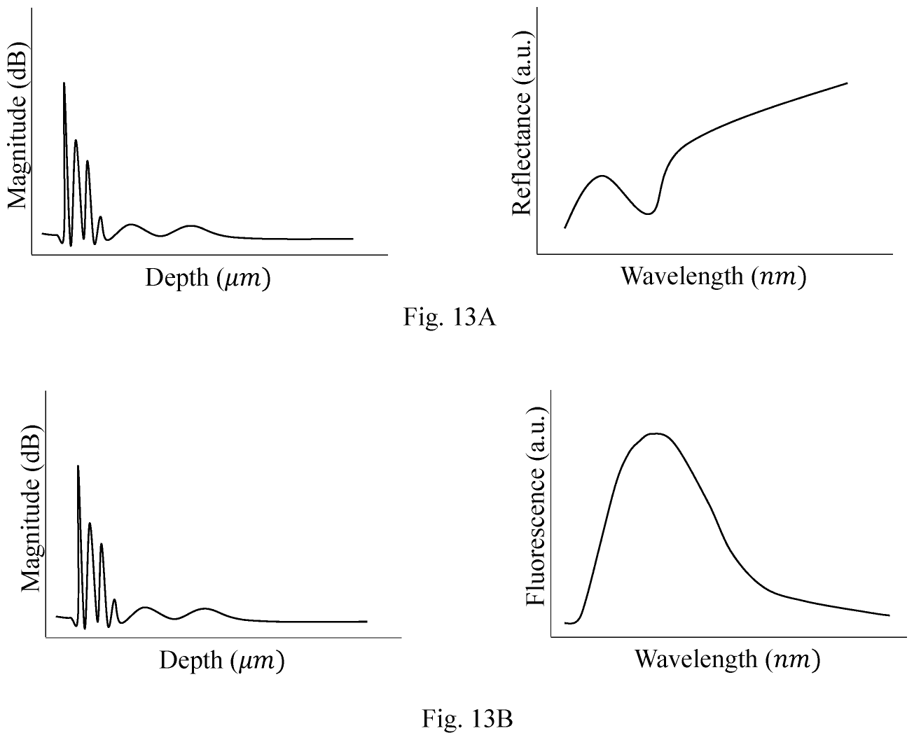

FIG. 13A illustrates example 1-D sample data sources for a multimodal feature detection algorithm in accordance with illustrative embodiments of the present disclosure. In this example, sample data inputs include a depth-dependent line (e.g., A-line) from an interferometric dataset (e.g., OCT) and a diffuse reflectance spectrum from a spectroscopy dataset (e.g., NIRS). Similarly, the spectroscopy dataset may be integrated over a detectors bandwidth and be provided by a single scalar value. The interferometric dataset and spectroscopic dataset may be input into a machine-learned algorithm as a single dataset (that is, appended to each other and then input) or may be used as separate inputs. In both cases, the interferometric dataset and spectroscopic dataset are considered as provided to the machine-learned algorithm.

FIG. 13B illustrates example 1-D sample data sources for a multimodal feature detection algorithm in accordance with illustrative embodiments of the present disclosure. In this example, sample data inputs include a depth-dependent line (e.g., A-line) from an interferometric dataset (e.g., OCT) and a fluorescence spectrum from a fluorescence spectroscopy dataset. Similarly, the spectroscopy dataset may be integrated over a detectors bandwidth and be provided by a single scalar value. The interferometric dataset and fluorescence dataset may be input into a machine-learned algorithm as a single dataset (that is, appended to each other and then input) or may be used as separate inputs. In both cases, the interferometric dataset and fluorescence dataset are considered as provided to the machine-learned algorithm.

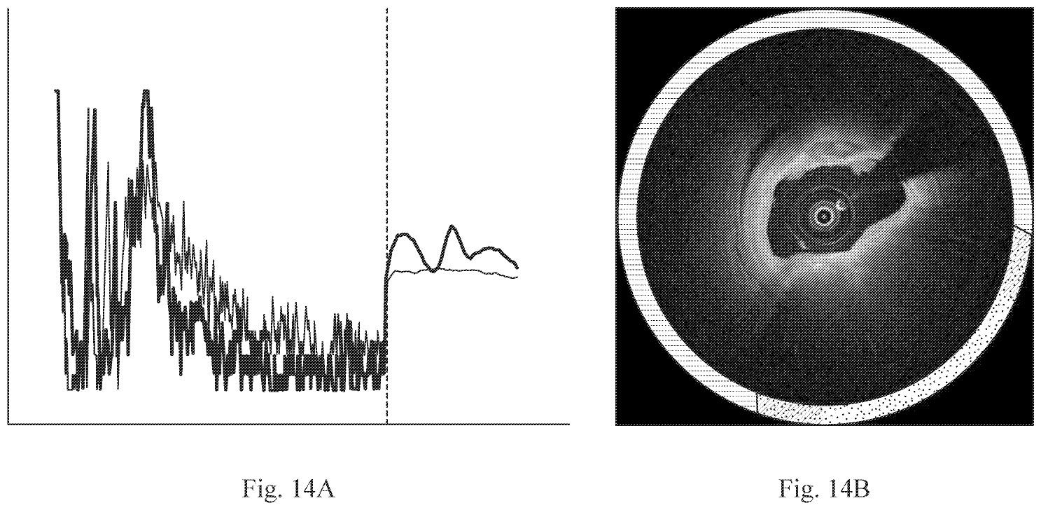

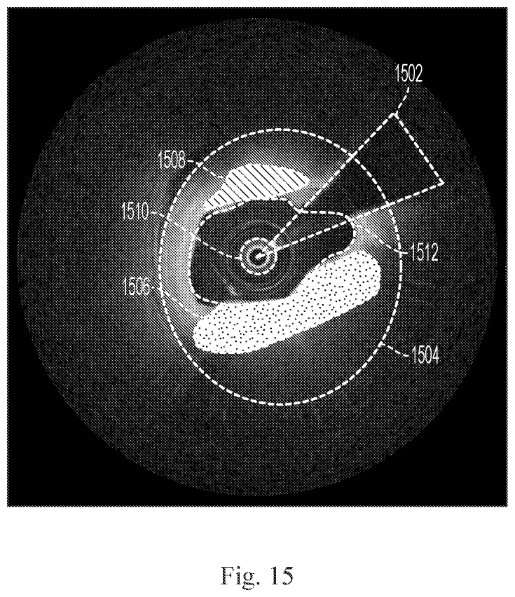

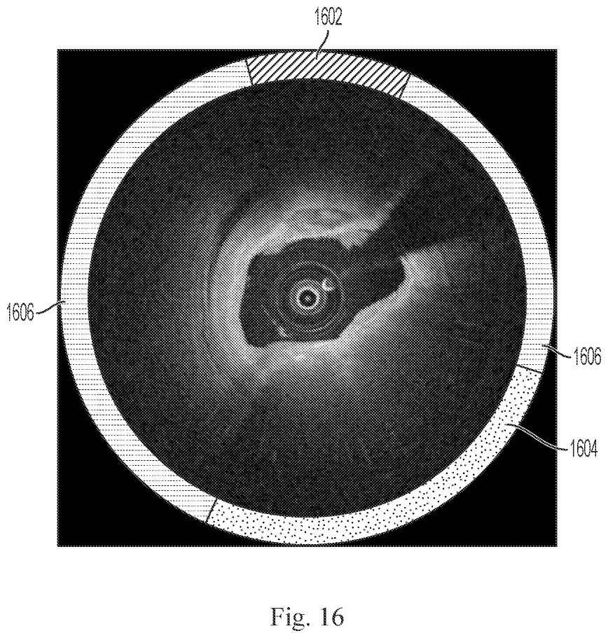

FIG. 14A illustrates example 1-D sample data sources for a multimodal feature detection algorithm appended together in accordance with illustrative embodiments of the present disclosure. In this example, sample data inputs include a depth-dependent line (e.g., A-line) from an interferometric dataset (e.g., OCT) and a reflectance spectrum from a diffuse reflectance spectroscopy dataset. The dashed vertical line represents the transition between the different sets (the point of appending). Similarly, the spectroscopy dataset may be integrated (e.g., over a detector's bandwidth) and be provided as a single scalar value (e.g., appended to A-line scan data from an interferometric dataset).

FIG. 14B illustrates example 2-D sample data sources for a multimodal feature detection algorithm in accordance with illustrative embodiments of the present disclosure. In this example, sample data inputs include a depth-dependent, rotationally acquired, image of an artery lumen wall displayed in Cartesian space (e.g., A-lines vs. rotational position) from an interferometric dataset (e.g., OCT) along with an array of spectra from diffuse reflectance spectroscopy measurements, also rotationally acquired from an artery lumen wall, co-registered to the first interferometric dataset. Similarly, the data could be input to a detection algorithm in polar space, or other coordinate system. The interferometric dataset and spectroscopic dataset may be input into a machine-learned algorithm as a single dataset (that is, appended to each other and then input) or may be used as separate inputs.

FIGS. 15-25 represent potential outputs from machine-learned algorithms that illustrate various embodiments of systems and methods disclosed herein. Different examples include detected features, feature representations, feature transformations, measurements, and combinations thereof. These examples are illustrative, not limiting as to what the output(s) of a machine-learned algorithm may look like, conceptually or literally.

FIG. 15 represents example segmentation outputs overlaid on and oriented with respect to an input 2-D arterial OCT image with the example outputs being generated (e.g., predicted) using a pre-trained multimodal feature detection algorithm (an exemplary machine-learned algorithm) in accordance with illustrative embodiments of the present disclosure. In this example, sample data inputs include a depth-dependent, rotationally acquired, image of an artery lumen wall displayed in Cartesian space (e.g., A-lines vs. rotational position) from an OCT dataset along with an array of spectra from diffuse reflectance spectroscopy measurements, also rotationally acquired from an artery lumen wall, co-registered to the OCT dataset. The co-registered OCT sample data and spectroscopy sample data would be provided to the machine-learned algorithm (e.g., as input) (e.g., after being appended to each other). Exemplary detected outputs from the machine-learned algorithm shown include feature representations of a guide wire 1502, the external elastic membrane (EEM) 1504, a lipid pool 1506, a calcium deposit 1508, the catheter sheath location 1510, and the inner lumen of the artery 1512, overlaid with respect to the OCT image of the artery wall derived from the OCT dataset. The example outputs may be displayed via a graphical user interface on a display.

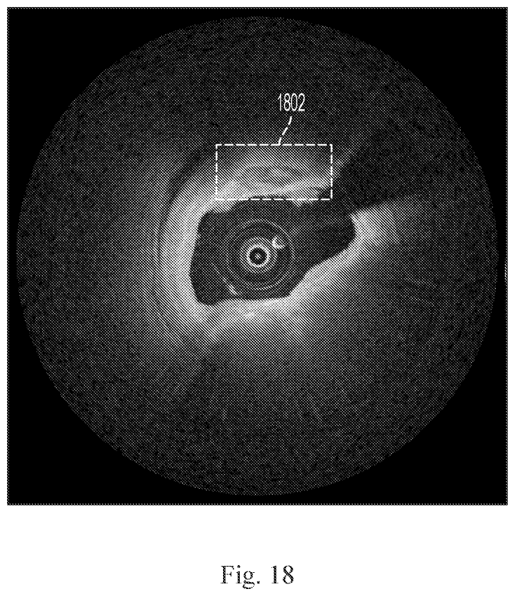

FIG. 16 represents example line-based segmentation outputs oriented with respect to an input 2-D arterial OCT image with the example outputs being generated (e.g., predicted) using a pre-trained multimodal feature detection algorithm (an exemplary machine-learned algorithm) in accordance with illustrative embodiments of the present disclosure. In this example, sample data inputs include a depth-dependent, rotationally acquired, image of an artery lumen wall displayed in Cartesian space (e.g., A-lines vs. rotational position) from an OCT dataset along with an array of spectra from diffuse reflectance spectroscopy measurements, also rotationally acquired from an artery lumen wall, co-registered to the OCT dataset. The co-registered OCT sample data and spectroscopy sample data would be provided to the machine-learned algorithm (e.g., as input) (e.g., after being appended to each other). Exemplary detected outputs from the machine-learned algorithm shown include a feature representation of a calcium deposit 1602, a lipid pool 1604, and no pathology 1606 overlaid with respect to the OCT image of the artery wall derived from the OCT dataset. In some embodiments, the particular coloring of one or more the feature representations may be indicative of one or more measurements determined with the machine-learned algorithm (e.g., the machine-learned algorithm may be a feature detection and measurement algorithm). For example, the shade of color (e.g., red and/or yellow) may be indicative of a measurement related to the feature of interest represented by the feature representation. For example, different shades of yellow may indicate different cap thicknesses (e.g., based on bucketing a value into one of a plurality of ranges). The example outputs may be displayed via a graphical user interface on a display.

FIG. 17 represents example line-based segmentation outputs oriented with respect to an input 2-D arterial OCT image with the example outputs being generated (e.g., predicted) using a pre-trained multimodal feature detection algorithm (an exemplary machine-learned algorithm) in accordance with illustrative embodiments of the present disclosure. In this example, sample data inputs include a depth-dependent, rotationally acquired, image of an artery lumen wall displayed in Cartesian space (e.g., A-lines vs. rotational position) from an OCT dataset along with an array of spectra from NIRS measurements, also rotationally acquired from an artery lumen wall, co-registered to the OCT dataset. The co-registered OCT sample data and spectroscopy sample data would be provided to the machine-learned algorithm (e.g., as input) (e.g., after being appended to each other). Exemplary detected outputs from the machine-learned algorithm shown include feature representations of a calcium deposit 1602, a lipid pool with a specific molecular composition 1604a, a lipid pool with a different molecular composition from the first 1604b, and no pathology 1606 overlaid with respect to the OCT image of the artery wall derived from the OCT dataset. Molecular composition may be determined using one or more measurements, for example determined using the machine-learned algorithm (e.g., the machine-learned algorithm may be a feature detection and measurement algorithm). The example outputs may be displayed via a graphical user interface on a display.

FIG. 18 represents example bounding-boxed based segmentation outputs oriented with respect to an input 2-D arterial OCT image with the example outputs being generated (e.g., predicted) using a pre-trained multimodal feature detection algorithm (an exemplary machine-learned algorithm) in accordance with illustrative embodiments of the present disclosure. In this example, sample data inputs include a depth-dependent, rotationally acquired, image of an artery lumen wall displayed in Cartesian space (e.g., A-lines vs. rotational position) from an OCT dataset along with an integrated reflectance intensity measurement, also rotationally acquired from an artery lumen wall, co-registered to the OCT dataset. The co-registered OCT sample data and spectroscopy sample data would be provided to the machine-learned algorithm (e.g., as input) (e.g., after being appended to each other). Exemplary detected outputs from the machine-learned algorithm shown include a feature representation of a calcium deposit 1802 overlaid with respect to the OCT image of the artery wall derived from the OCT dataset. The example outputs may be displayed via a graphical user interface on a display.

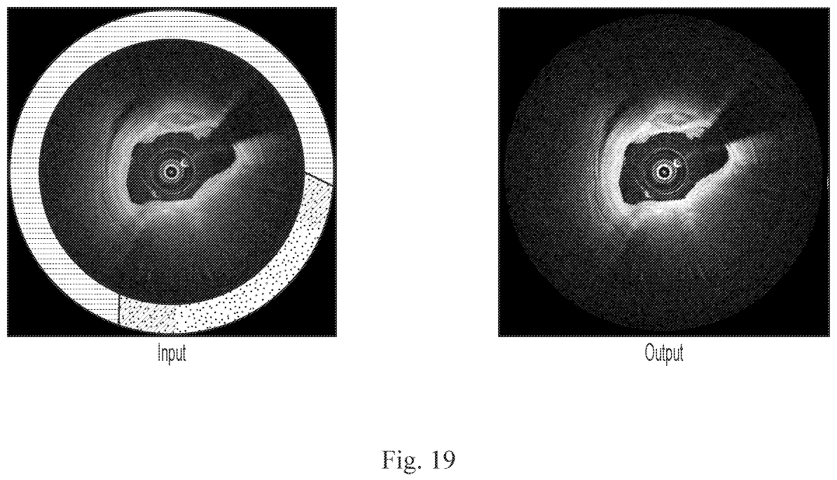

FIG. 19 represents an example output from a pre-trained multimodal feature enhancement algorithm (an exemplary machine-learned algorithm that transforms feature representations) in accordance with illustrative embodiments of the present disclosure. In this example, sample data inputs include a depth-dependent, rotationally acquired, image of an artery lumen wall displayed in Cartesian space (e.g., A-lines vs. rotational position) from an OCT dataset along with an array of spectra from spectroscopy measurements, also rotationally acquired from an artery lumen wall, co-registered with the OCT dataset. The co-registered OCT sample data and spectroscopy sample data would be provided to the machine-learned algorithm (e.g., as input) (e.g., after being appended to each other). Exemplary enhancements made by the machine-learned algorithm shown consist of a contrast enhanced OCT image (“Output”). The OCT image otherwise derived from the OCT dataset (shown in the “Input”) has contrast enhanced by the spectroscopic data input (illustrated in the outer ring of the “Input”). For example, where spectroscopy data indicates a feature of interest (e.g., a lipid pool), the presence of that feature in the spectroscopy data may be detected by the algorithm and then automatically transform, in this case enhance, the representation of the feature in the OCT image derived from the OCT dataset. In this case, for example, contrast of the lipid pool feature in the transformed OCT image has been enhanced by the machine-learned algorithm based on the spectroscopy data used as part of the input to the algorithm. The example output may be displayed via a graphical user interface on a display.

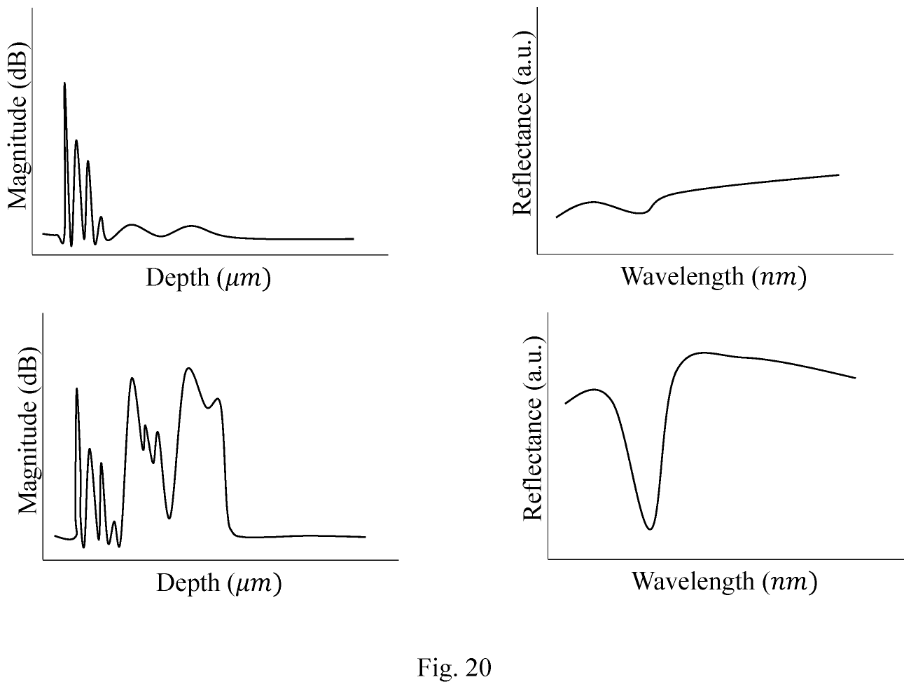

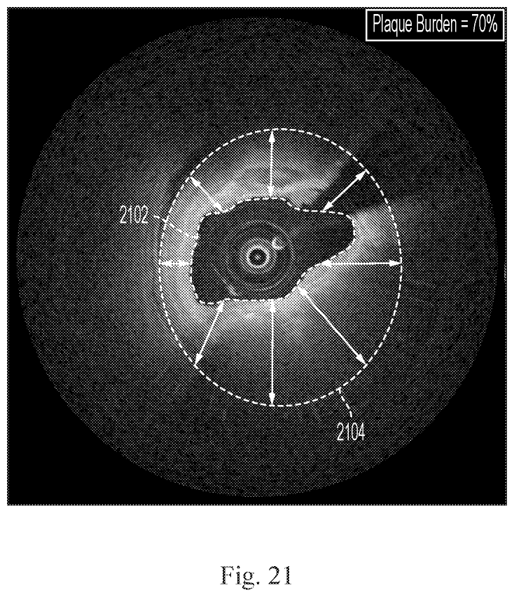

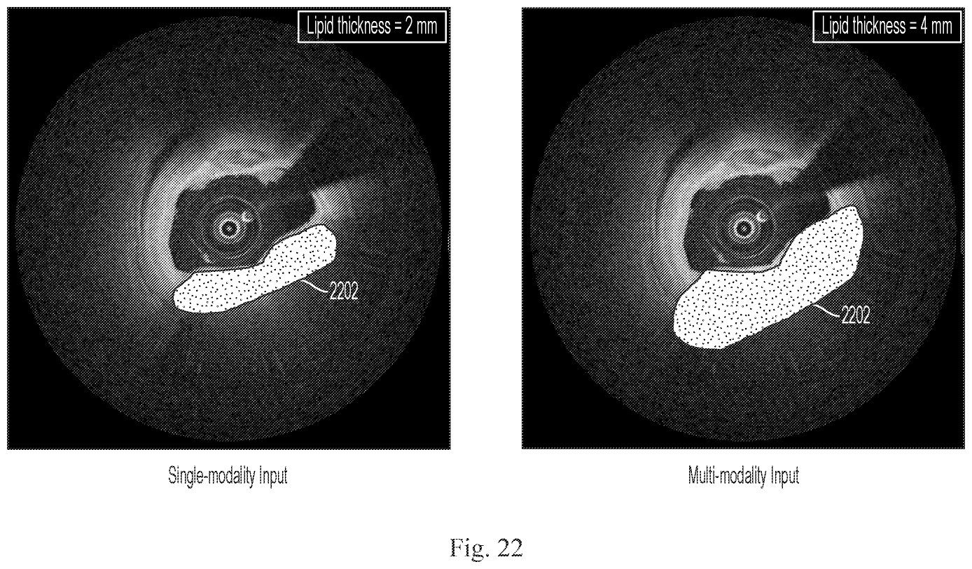

FIG. 20 represents an example output from a pre-trained multimodal feature enhancement algorithm (an exemplary machine-learned algorithm that transforms feature representations) in accordance with illustrative embodiments of the present disclosure. In this example, sample data inputs include a depth-dependent line (e.g., A-line) from an interferometric dataset (e.g., OCT) and a reflectance spectrum from a diffuse reflectance spectroscopy dataset. Exemplary enhancements made by the machine-learned algorithm shown consist of an enhanced (e.g., contrast, visibility) depth-dependent line as well as an enhanced (e.g., calibrated, scaled or normalized) reflectance spectrum. The OCT sample data and spectroscopy sample data would be provided to the machine-learned algorithm (e.g., as input) (e.g., after being appended to each other). That is, using the combined input of the interferometric dataset and the reflectance spectrum, the machine-learned algorithm can separately enhance both datasets. The example output may be displayed via a graphical user interface on a display. In this case, the transformation (e.g., enhancement) is performed in a reduced dimension as compared to the example of FIG. 19.

FIG. 21 represents an example output from a pre-trained multimodal feature detection and measurement algorithm (an exemplary machine-learned algorithm) in accordance with illustrative embodiments of the present disclosure. In this example, sample data inputs include a depth-dependent, rotationally acquired, image of an artery lumen wall displayed in Cartesian space (e.g., A-lines vs rotational position) from an OCT dataset along with an array of spectra from diffuse reflectance spectroscopy measurements, also rotationally acquired from an artery lumen wall, co-registered to the OCT dataset. The co-registered OCT sample data and spectroscopy sample data would be provided to the machine-learned algorithm (e.g., as input) (e.g., after being appended to each other). Exemplary feature detection outputs from the machine-learned algorithm include the inner lumen 2102 and EEM 2104, while exemplary measurement outputs from the machine-learned algorithm shown include a plaque burden index (in this case determined to be 70%). The arrows indicate spacing between the inner lumen and EEM (e.g., used by the machine-learned algorithm in determining the plaque burden index measurement). The example output may be displayed via a graphical user interface on a display.

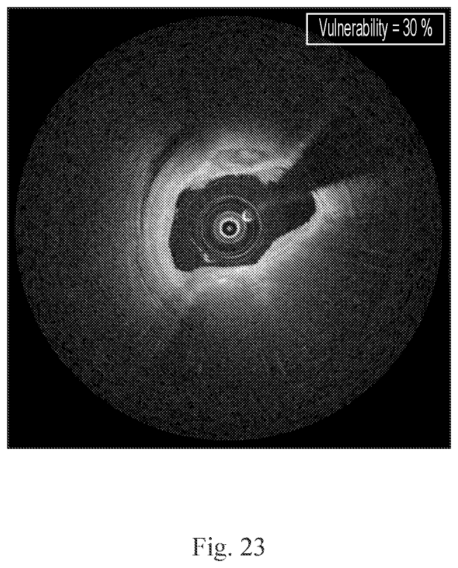

FIG. 22 represents an example output from a pre-trained multimodal feature detection and measurement algorithm (an exemplary machine-learned algorithm) in accordance with illustrative embodiments of the present disclosure. In this example, sample data inputs include a depth-dependent, rotationally acquired, image of an artery lumen wall displayed in Cartesian space (e.g., A-lines vs. rotational position) from an OCT dataset along with an array of spectra from diffuse spectroscopy measurements, also rotationally acquired from an artery lumen wall, co-registered to the OCT dataset. The co-registered OCT sample data and spectroscopy sample data would be provided to the machine-learned algorithm (e.g., as input) (e.g., after being appended to each other). Exemplary detection outputs include a detected lipid pool 2202, while exemplary measurement outputs shown include a lipid pool thickness. Multi-modality input (e.g., of the OCT dataset and spectroscopy dataset) may produce more accurate measurements. In this case, lipid pool thickness may be determined to be larger when considering both OCT data and spectroscopy data (“Multi-modality Input”) instead of OCT data alone (“Single-modality Input”). The example output may be displayed via a graphical user interface on a display.

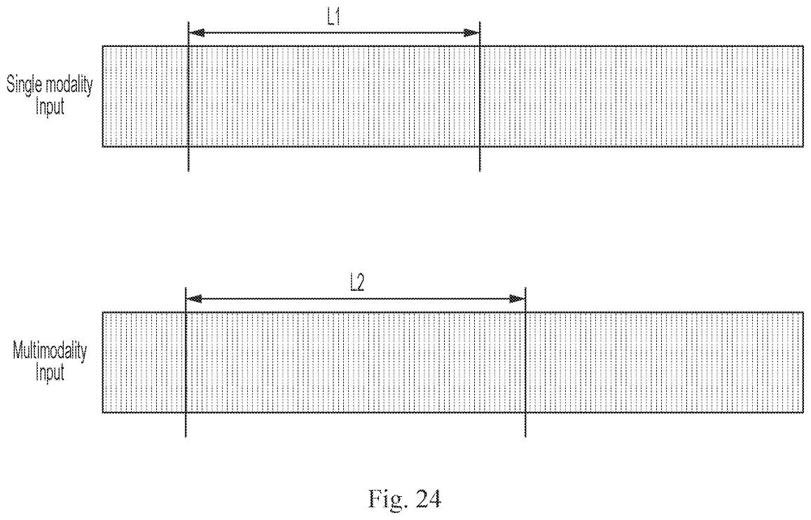

FIG. 23 represents an example output from a pre-trained multimodal feature detection and measurement algorithm (an exemplary machine-learned algorithm) in accordance with illustrative embodiments of the present disclosure. In this example, sample data inputs include a depth-dependent, rotationally acquired, image of an artery lumen wall displayed in Cartesian space (e.g., A-lines vs. rotational position) from an OCT dataset along with an array of spectra from diffuse spectroscopy measurements, also rotationally acquired from an artery lumen wall, co-registered to the OCT dataset. The OCT sample data and spectroscopy sample data would be provided to the machine-learned algorithm (e.g., as input) (e.g., after being appended to each other). Exemplary measurement outputs from the machine-learned algorithm include a plaque vulnerability (e.g., risk of rupture) index. In this example, no feature representations (transformed or untransformed) are output; only the plaque vulnerability measurement. The OCT image derived from the original OCT dataset is shown. The example output may be displayed via a graphical user interface on a display.

FIG. 24 represents an example output from a pre-trained multimodal feature detection and measurement algorithm (an exemplary machine-learned algorithm) in accordance with illustrative embodiments of the present disclosure. In this example, sample data inputs include a depth-dependent, rotationally acquired, image of an artery lumen wall displayed in Cartesian space (e.g., A-lines vs. rotational position) from an OCT dataset along with an array of spectra from diffuse spectroscopy measurements, also rotationally acquired from an artery lumen wall, co-registered to the OCT dataset. The OCT sample data and spectroscopy sample data would be provided to the machine-learned algorithm (e.g., as input) (e.g., after being appended to each other). Exemplary measurement outputs from the machine-learned algorithm include an improved automatic stent placement suggestion prediction with a longer stentable region (L2>L1) (where the larger grey box represents the artery), compared to a single modality algorithm, due to, for example, improved detection of the extent of a necrotic core plaque and based on the knowledge that the algorithm has been trained on that it is undesirable to have the edge of a stent placed on a necrotic core. In some embodiments, a visualization of a stent placement suggestion may include a visual indicator useful in understanding physical location along the artery. The example output may be displayed via a graphical user interface on a display.

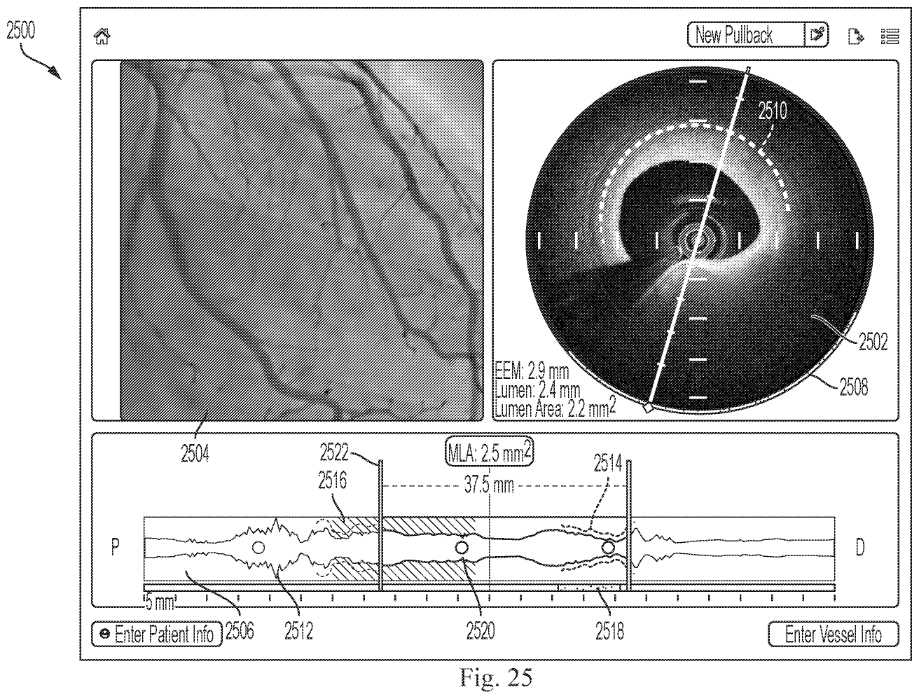

FIG. 25 illustrates an example display (e.g., graphical user interface(s)) of outputs from a pre-trained multimodal feature detection and measurement algorithm (an exemplary machine-learned algorithm) in accordance with illustrative embodiments of the present disclosure. In this example, sample data inputs may include a depth-dependent, rotationally acquired, image of an artery lumen wall displayed in Cartesian space (e.g., A-lines vs. rotational position) from an OCT dataset along with an array of spectra from diffuse spectroscopy measurements, also rotationally acquired from an artery lumen wall, co-registered to the OCT dataset. The OCT sample data and spectroscopy sample data would be provided to the machine-learned algorithm (e.g., as input) (e.g., after being appended to each other). Shown in the multi-panel UI 2500 is an arterial OCT image 2502, a 2D angiography projection 2504, a longitudinal pullback representation of multiple 2D imaging positions along a catheter pullback 2506. Example outputs from the feature detection algorithm include a lipid arc 2508 and an EEM arc 2510 overlaid on the OCT image derived from the OCT dataset, a frame-based lumen 2512, EEM 2514, calcium 2516, lipid 2518 and side branch 2520 detected feature representations along the imaging pullback representation. Finally, an automatically predicted measurement for optimal stent placement location 2522 based on the multimodal feature detection algorithm.

Methods disclosed herein can be used for automated stent placement planning. In some embodiments, EEM cannot be located because there is too much diseased tissue to place stent edge. In some embodiments, it is undesirable for a stent to be disposed directly on a lipid pool/necrotic core. In some embodiments, calcium presence means an area needs to be prepped before stenting but the presence of calcium does not necessarily decide stent position. Because accuracy of EEM measurements, lipid pool/necrotic core measurements, and/or calcium detection can be potentially improved using methods disclosed herein, stent placement planning can be improved in some embodiments. Because feature detection may occur automatically, stent placement planning can be performed automatically as well (e.g., based on optimizing one or more measurements, e.g. using detected feature(s)).

In some embodiments, a physiological measurement is made, for example with a machine-learned algorithm. Examples of such physiological measurements that may be made include flow rate, fractional flow reserve, pressure drop, absolute or relative coronary flow (CF), fractional flow reserve (FFR), instantaneous wave free ratio/resting full cycle ratio (iFR/RFR), index of microcirculatory resistance (IMR), hyperemic microvascular resistance (HMR), hyperemic stenosis resistance (HSR), and coronary flow reserve (CFR). Combinations of such physiological measurements may be also made. In some embodiments, a physiology measurement may be based at least partially on a geometric measurement and/or a measurement of image-dynamics (e.g., speckle variance) (e.g., of one or more images from an interferometric modality). In some embodiments, data from a first characterization modality [e.g., a interferometric modality (e.g., OCT)] may be used to calculate a measurement that is a physiological measurement, for example by using the data as an input to a machine-learned algorithm where the measurement is an output. In some embodiments, a physiological measurement is determined automatically (e.g., by a processor) from data from a first characterization modality (e.g., OCT), for example using a machine-learned algorithm. In some embodiments, data from a second modality [e.g., a spectroscopic modality (e.g., NIRS)] may be used to improve (e.g., more accurately reflect physical reality) a physiology measurement made with data from at least a first characterization modality. Such improvement may be made by, for example, detecting and/or characterizing an object present in a person's physiology. For example, in some embodiments, a physiological measurement is improved by detecting and/or characterizing an object present in a wall of an artery. For example, a physiological measurement may be different when a lipid-rich plaque is detected as compared to when a calcified plaque is detected in its place. A physiological measurement may be affected by wall characteristics (e.g., wall strength, shear stress, friction) of an artery (e.g., at the location of a detected plaque). Detecting and incorporating information about one or more objects within an artery wall can therefore improve a physiology measurement related to that artery. In some embodiments, data from two modalities may be used by a machine-learned algorithm to calculate a physiology measurement (e.g., with higher accuracy). Such physiological measurements can be improved as compared to if the same measurement was made using sample data for a characterization modality of a subject without accounting for the presence and/or character of an object present in the physiology of the subject.

In some embodiments, data from at least a first characterization modality [e.g., an interferometric modality (e.g., OCT)] is used to make a physiological measurement (e.g., in combination with data from a second characterization modality [e.g., a spectroscopic modality (e.g., NIRS)]). Data from at least one of a first characterization modality and a second characterization modality may be used to improve the measurement. Such data used for improvement may correspond to a different feature than the feature for which the physiological measurement is made. For example, data from a first characterization modality may be used to make a physiological measurement and the measurement may be improved using one or more other measurements and/or information about one or more detected features, such as, for example, location and/or characterization of a plaque. The one or more other measurements and/or one or more detected features may be determined using data (e.g., other data) from the first characterization modality and/or a second characterization modality. Such a physiological measurement and/or one or more other measurements and/or one or more detected features may be determined using a machine-learned algorithm as disclosed herein.

Accordingly, in some embodiments, co-registered data for at least two characterization modalities [e.g., an interferometric modality and a spectroscopic modality (e.g., OCT and NIRS)] are collected during a pullback of a catheter, fed into a machine-learned algorithm as input, and a physiological measurement is output from the algorithm. The physiological measurement can be improved as compared to if the same measurement was made using solely the data from the first characterization modality. In some embodiments, data from only one characterization modality is input into a machine-learned algorithm that has been trained using training labels derived from a second characterization modality and a physiological measurement is output (e.g., that is improved as compared to if the same measurement was made using solely the data from the first characterization modality). Improvement of a physiological measurement, such as, for example, flow, pressure drop, resistance, or the like, may be realized by accounting for location and/or composition of a plaque and/or curvature of a vessel (e.g., artery). Location and/or composition of plaque may have an effect on, for example, wall strength and/or wall friction that influences physiology and therefore accounting for the plaque may improve an ultimate physiological measurement. A machine-learned algorithm may be trained to learn a relationship between physiology (e.g., plaque location and/or composition) and physiological measurement for use in making further physiological measurements using sample data.

Using methods disclosed herein, data from one modality can be used to evaluate NURD of a catheter, and that measurement output can then be used to correct NURD in data (e.g., image(s)) for a second modality. For example, a machine-learned algorithm may take as input first sample data from a first characterization modality and second sample data from a second characterization modality. The machine-learned algorithm may be trained to evaluate NURD with respect to the first characterization modality. That will naturally involve using the first sample data, though the first sample data may (or may not) transformed (e.g., enhanced) by the machine-learned algorithm using the second sample data before evaluating NURD. The measurement output of the machine-learned algorithm may be one or more discrete measurements reflective of the NURD of the catheter. The measurement output may then be used to transform (e.g., enhance) the second sample data to mitigate the effects of NURD that would otherwise present. This process may all occur automatically as part of the machine-learned algorithm such that the first sample data and the second sample data are provided to the machine-learned algorithm (e.g., along with any other required input, such as, for example, characteristics of the catheter and/or pullback used to generate the data) and the machine-learned algorithm outputs a “NURD-corrected” image, or feature representation(s), corresponding to one of the characterization modalities based on another of the characterization modalities.

Using methods disclosed herein, spectroscopy-sensitive markers on a sheath of a probe (e.g., catheter) can be detected to improve (e.g., reduce) NURD on one or more images (e.g., OCT image(s)). For example, fiducials on a sheath that produce a signal on a spectroscopy channel but are transparent in OCT data can be detected using methods disclosed herein. Fiducial and/or spectroscopy data can be used to determine NURD and perform correction on a depth-dependent modality image (e.g., an OCT image). In some embodiments, a method, such a feature detection method, is performed prior to pullback of a catheter with which first sample data from a first characterization modality and second sample data from a second characterization modality are acquired.

A bad frame, insufficient blood flushing, contrast injection detection, or a combination thereof may be detected using methods disclosed herein, for example automatically using a machine-learned algorithm. Blood flush can be detected using methods and systems disclosed herein to (e.g., automatically) initiate start of pullback and/or start of scanning for a probe (e.g., a catheter). For example, first sample data from a first characterization modality and/or second sample data from a second characterization modality may be provided to a machine-learned algorithm trained to determine one or more measurements indicative of a bad frame, insufficient blood flushing, contrast injection detection, or a combination thereof. In some embodiments, simple thresholds may be used for the one or more measurements determined by the machine-learned algorithm to determine a bad frame, insufficient blood flushing, contrast injection detection, or a combination thereof.

Using methods disclosed herein, an optical probe break or poor transmission can be detected. For example, first sample data from a first characterization modality and/or second sample data from a second characterization modality may be provided to a machine-learned algorithm trained to determine one or more measurements indicative of an optical probe break and/or poor transmission. The machine-learned algorithm may output a determination of an optical probe break and/or poor transmission (e.g., automatically) and/or one or more measurements for review by a user for the user to assess whether an optical probe break and/or poor transmission is occurring or has occurred.

In some embodiments, one modality is used to dynamically measure a system or catheter transfer function, and that data is used to correct or improve quality of a second modality. In some embodiments, one modality is used to identify structures or defects in a catheter, and that detection is used to mask out valid/invalid regions for a second modality based on the identified structures or defects.

“Imaged” refers to detection of light (e.g., at a given spatial position). For example, detecting light from a specific position on a sample using a waveguide is referred to as “imaging” the sample at that position. Imaging may be performed using, for example, depth dependent imaging, such as OCT, ultrasound, or variable confocal imaging. A spectroscopic modality may be DRS, fluorescence, auto-fluorescence, spontaneous Raman spectroscopy, coherent Raman spectroscopy, hyperspectral imaging, or a point measurement at a specified wavelength. A characterization modality may use, for example, spectrally separated detectors or large bandwidth integration.

Various embodiments of the present disclosure may use data from any suitable characterization modality. Generally, data from at least two characterization modalities are used, though data from more than two may be used in some embodiments. A characterization modality (e.g., first and/or second modality/modalities) may be a spatially resolved imaging modality, such as, for example, a depth-dependent imaging modality. In some embodiments, a characterization modality is OCT, ultrasound, or variable confocal imaging; combinations thereof may be used, though generally would be at least partially redundant. A characterization modality may be a tomography modality. A characterization modality may be a microscopy modality. A characterization modality may be an interferometric modality. A characterization modality may be a spectroscopic modality. A spectroscopic modality may be a diffuse spectroscopy modality. For example, a characterization modality may be DRS, fluorescence, auto-fluorescence, spontaneous Raman spectroscopy, coherent Raman spectroscopy, hyperspectral imaging, or a point measurement at a specified wavelength. A characterization modality may be an intraluminal characterization modality that is suitable to characterize a lumen of a subject. A characterization modality may be a vascular characterization modality that is suitable to characterize a structure of a vascular system of a subject. In certain embodiments, data from a depth-dependent imaging modality and spectroscopic modality are used together (e.g., provided to a machine-learned algorithm).

The term “image,” for example, as in a two- or three-dimensional image of a sample, includes any visual representation, such as a photo, a video frame, streaming video, as well as any electronic, digital, or mathematical analogue of a photo, video frame, or streaming video. Thus, “image” may refer to data that could be, but is not necessarily, displayed. Any system or apparatus described herein, in certain embodiments, includes a display for displaying an image or any other result produced by a processor. Any method described herein, in certain embodiments, includes a step of displaying an image or any other result produced by the method. Any system or apparatus described herein, in certain embodiments, outputs an image to a remote receiving device [e.g., a cloud server, a remote monitor, or a hospital information system (e.g., a picture archiving and communication system (PACS))] or to an external storage device that can be connected to the system or to the apparatus. In some embodiments, an image is produced using a multimodal catheter (e.g., including an interferometric imaging modality and a spectroscopic imaging modality). In some embodiments, an image is a two-dimensional (2D) image. In some embodiments, an image is a three-dimensional (3D) image. In some embodiments, an image is a reconstructed image. An image (e.g., a 3D image) may be a single image or a set of images.

Tissue volume refers to the volume of tissue seen by a detection waveguide (e.g., due to its numerical aperture). Depending on optical geometry, tissue volume can change based on the distance of a probe (e.g., catheter) to a sample. A probe may be a catheter, such as, for example, a cardiac catheter.

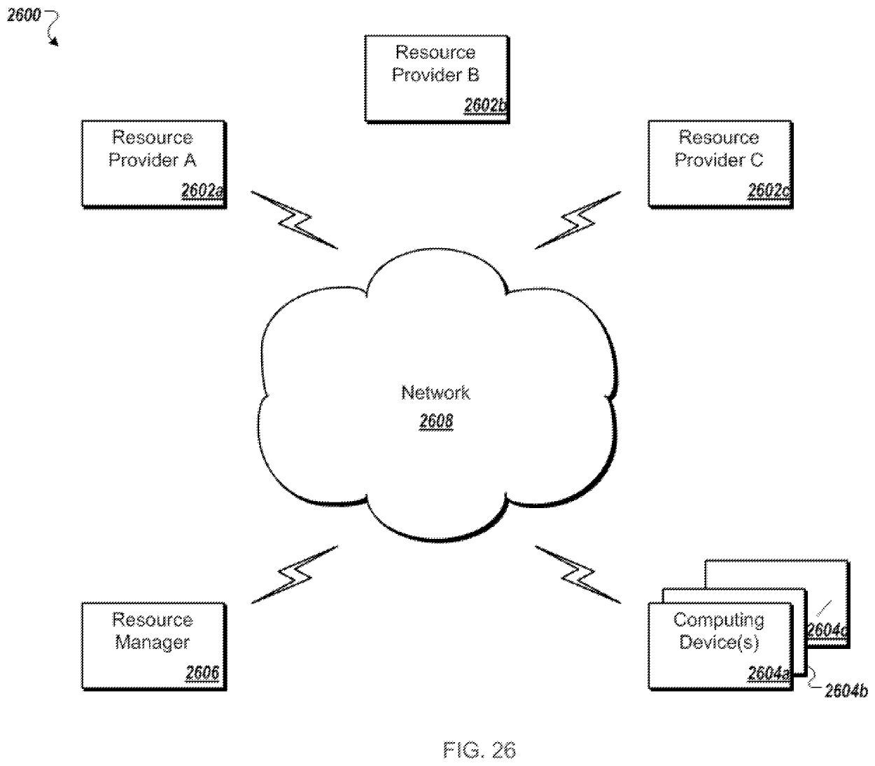

Illustrative embodiments of systems and methods disclosed herein were described above with reference to computations performed locally by a computing device. However, computations performed over a network are also contemplated. FIG. 26 shows an illustrative network environment 2600 for use in the methods and systems described herein. In brief overview, referring now to FIG. 26, a block diagram of an illustrative cloud computing environment 2600 is shown and described. The cloud computing environment 2600 may include one or more resource providers 2602a, 2602b, 2602c (collectively, 2602). Each resource provider 2602 may include computing resources. In some implementations, computing resources may include any hardware and/or software used to process data. For example, computing resources may include hardware and/or software capable of executing algorithms, computer programs, and/or computer applications. In some implementations, illustrative computing resources may include application servers and/or databases with storage and retrieval capabilities. Each resource provider 2602 may be connected to any other resource provider 2602 in the cloud computing environment 2600. In some implementations, the resource providers 2602 may be connected over a computer network 2608. Each resource provider 2602 may be connected to one or more computing device 2604a, 2604b, 2604c (collectively, 2604), over the computer network 2608.

The cloud computing environment 2600 may include a resource manager 2606. The resource manager 2606 may be connected to the resource providers 2602 and the computing devices 2604 over the computer network 2608. In some implementations, the resource manager 2606 may facilitate the provision of computing resources by one or more resource providers 2602 to one or more computing devices 2604. The resource manager 2606 may receive a request for a computing resource from a particular computing device 2604. The resource manager 2606 may identify one or more resource providers 2602 capable of providing the computing resource requested by the computing device 2604. The resource manager 2606 may select a resource provider 2602 to provide the computing resource. The resource manager 2606 may facilitate a connection between the resource provider 2602 and a particular computing device 2604. In some implementations, the resource manager 2606 may establish a connection between a particular resource provider 2602 and a particular computing device 2604. In some implementations, the resource manager 2606 may redirect a particular computing device 2604 to a particular resource provider 2602 with the requested computing resource.

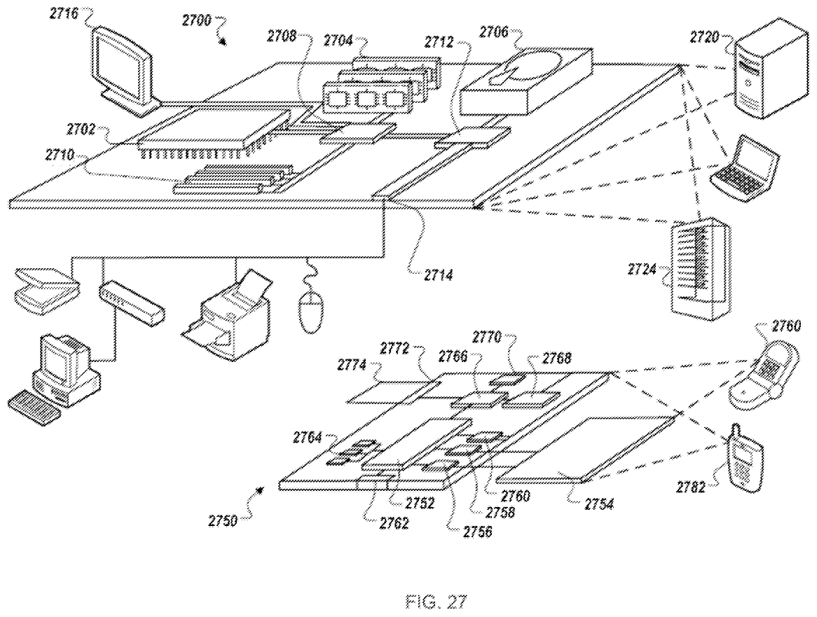

FIG. 27 shows an example of a computing device 2700 and a mobile computing device 2750 that can be used in the methods and systems described in this disclosure. The computing device 2700 is intended to represent various forms of digital computers, such as laptops, desktops, workstations, personal digital assistants, servers, blade servers, mainframes, and other appropriate computers. The mobile computing device 2750 is intended to represent various forms of mobile devices, such as personal digital assistants, cellular telephones, smart-phones, and other similar computing devices. The components shown here, their connections and relationships, and their functions, are meant to be examples only, and are not meant to be limiting.

The computing device 2700 includes a processor 2702, a memory 2704, a storage device 2706, a high-speed interface 2708 connecting to the memory 2704 and multiple high-speed expansion ports 2710, and a low-speed interface 2712 connecting to a low-speed expansion port 2714 and the storage device 2706. Each of the processor 2702, the memory 2704, the storage device 2706, the high-speed interface 2708, the high-speed expansion ports 2710, and the low-speed interface 2712, are interconnected using various busses, and may be mounted on a common motherboard or in other manners as appropriate. The processor 2702 can process instructions for execution within the computing device 2700, including instructions stored in the memory 2704 or on the storage device 2706 to display graphical information for a GUI on an external input/output device, such as a display 2716 coupled to the high-speed interface 2708. In other implementations, multiple processors and/or multiple buses may be used, as appropriate, along with multiple memories and types of memory. Also, multiple computing devices may be connected, with each device providing portions of the necessary operations (e.g., as a server bank, a group of blade servers, or a multi-processor system). Also, multiple computing devices may be connected, with each device providing portions of the necessary operations (e.g., as a server bank, a group of blade servers, or a multi-processor system). Thus, as the term is used herein, where a plurality of functions are described as being performed by “a processor”, this encompasses embodiments wherein the plurality of functions are performed by any number of processors (e.g., one or more processors) of any number of computing devices (e.g., one or more computing devices). Furthermore, where a function is described as being performed by “a processor”, this encompasses embodiments wherein the function is performed by any number of processors (e.g., one or more processors) of any number of computing devices (e.g., one or more computing devices) (e.g., in a distributed computing system).

The memory 2704 stores information within the computing device 2700. In some implementations, the memory 2704 is a volatile memory unit or units. In some implementations, the memory 2704 is a non-volatile memory unit or units. The memory 2704 may also be another form of computer-readable medium, such as a magnetic or optical disk.

The storage device 2706 is capable of providing mass storage for the computing device 2700. In some implementations, the storage device 2706 may be or contain a computer-readable medium, such as a floppy disk device, a hard disk device, an optical disk device, or a tape device, a flash memory or other similar solid state memory device, or an array of devices, including devices in a storage area network or other configurations. Instructions can be stored in an information carrier. The instructions, when executed by one or more processing devices (for example, processor 2702), perform one or more methods, such as those described above. The instructions can also be stored by one or more storage devices such as computer- or machine-readable mediums (for example, the memory 2704, the storage device 2706, or memory on the processor 2702).

The high-speed interface 2708 manages bandwidth-intensive operations for the computing device 2700, while the low-speed interface 2712 manages lower bandwidth-intensive operations. Such allocation of functions is an example only. In some implementations, the high-speed interface 2708 is coupled to the memory 2704, the display 2716 (e.g., through a graphics processor or accelerator), and to the high-speed expansion ports 2710, which may accept various expansion cards (not shown). In the implementation, the low-speed interface 2712 is coupled to the storage device 2706 and the low-speed expansion port 2714. The low-speed expansion port 2714, which may include various communication ports (e.g., USB, Bluetooth®, Ethernet, wireless Ethernet) may be coupled to one or more input/output devices, such as a keyboard, a pointing device, a scanner, or a networking device such as a switch or router, e.g., through a network adapter.

The computing device 2700 may be implemented in a number of different forms, as shown in the figure. For example, it may be implemented as a standard server 2720, or multiple times in a group of such servers. In addition, it may be implemented in a personal computer such as a laptop computer 2722. It may also be implemented as part of a rack server system 2724. Alternatively, components from the computing device 2700 may be combined with other components in a mobile device (not shown), such as a mobile computing device 2750. Each of such devices may contain one or more of the computing device 2700 and the mobile computing device 2750, and an entire system may be made up of multiple computing devices communicating with each other.

The mobile computing device 2750 includes a processor 2752, a memory 2764, an input/output device such as a display 2754, a communication interface 2766, and a transceiver 2768, among other components. The mobile computing device 2750 may also be provided with a storage device, such as a micro-drive or other device, to provide additional storage. Each of the processor 2752, the memory 2764, the display 2754, the communication interface 2766, and the transceiver 2768, are interconnected using various buses, and several of the components may be mounted on a common motherboard or in other manners as appropriate.

The processor 2752 can execute instructions within the mobile computing device 2750, including instructions stored in the memory 2764. The processor 2752 may be implemented as a chipset of chips that include separate and multiple analog and digital processors. The processor 2752 may provide, for example, for coordination of the other components of the mobile computing device 2750, such as control of user interfaces, applications run by the mobile computing device 2750, and wireless communication by the mobile computing device 2750.

The processor 2752 may communicate with a user through a control interface 2758 and a display interface 2756 coupled to the display 2754. The display 2754 may be, for example, a TFT (Thin-Film-Transistor Liquid Crystal Display) display or an OLED (Organic Light Emitting Diode) display, or other appropriate display technology. The display interface 2756 may comprise appropriate circuitry for driving the display 2754 to present graphical and other information to a user. The control interface 2758 may receive commands from a user and convert them for submission to the processor 2752. In addition, an external interface 2762 may provide communication with the processor 2752, so as to enable near area communication of the mobile computing device 2750 with other devices. The external interface 2762 may provide, for example, for wired communication in some implementations, or for wireless communication in other implementations, and multiple interfaces may also be used.

The memory 2764 stores information within the mobile computing device 2750. The memory 2764 can be implemented as one or more of a computer-readable medium or media, a volatile memory unit or units, or a non-volatile memory unit or units. An expansion memory 2774 may also be provided and connected to the mobile computing device 2750 through an expansion interface 2772, which may include, for example, a SIMM (Single In Line Memory Module) card interface. The expansion memory 2774 may provide extra storage space for the mobile computing device 2750, or may also store applications or other information for the mobile computing device 2750. Specifically, the expansion memory 2774 may include instructions to carry out or supplement the processes described above, and may include secure information also. Thus, for example, the expansion memory 2774 may be provided as a security module for the mobile computing device 2750, and may be programmed with instructions that permit secure use of the mobile computing device 2750. In addition, secure applications may be provided via the SIMM cards, along with additional information, such as placing identifying information on the SIMM card in a non-hackable manner.

The memory may include, for example, flash memory and/or NVRAM memory (non-volatile random access memory), as discussed below. In some implementations, instructions are stored in an information carrier and, when executed by one or more processing devices (for example, processor 2752), perform one or more methods, such as those described above. The instructions can also be stored by one or more storage devices, such as one or more computer- or machine-readable mediums (for example, the memory 2764, the expansion memory 2774, or memory on the processor 2752). In some implementations, the instructions can be received in a propagated signal, for example, over the transceiver 2768 or the external interface 2762.

The mobile computing device 2750 may communicate wirelessly through the communication interface 2766, which may include digital signal processing circuitry where necessary. The communication interface 2766 may provide for communications under various modes or protocols, such as GSM voice calls (Global System for Mobile communications), SMS (Short Message Service), EMS (Enhanced Messaging Service), or MMS messaging (Multimedia Messaging Service), CDMA (code division multiple access), TDMA (time division multiple access), PDC (Personal Digital Cellular), WCDMA (Wideband Code Division Multiple Access), CDMA2000, or GPRS (General Packet Radio Service), among others. Such communication may occur, for example, through the transceiver 2768 using a radio-frequency. In addition, short-range communication may occur, such as using a Bluetooth®, Wi-Fi™, or other such transceiver (not shown). In addition, a GPS (Global Positioning System) receiver module 2770 may provide additional navigation- and location-related wireless data to the mobile computing device 2750, which may be used as appropriate by applications running on the mobile computing device 2750.

The mobile computing device 2750 may also communicate audibly using an audio codec 2760, which may receive spoken information from a user and convert it to usable digital information. The audio codec 2760 may likewise generate audible sound for a user, such as through a speaker, e.g., in a handset of the mobile computing device 2750. Such sound may include sound from voice telephone calls, may include recorded sound (e.g., voice messages, music files, etc.) and may also include sound generated by applications operating on the mobile computing device 2750.

The mobile computing device 2750 may be implemented in a number of different forms, as shown in the figure. For example, it may be implemented as a cellular telephone 2780. It may also be implemented as part of a smart-phone 2782, personal digital assistant, or other similar mobile device.

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms machine-readable medium and computer-readable medium refer to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term machine-readable signal refers to any signal used to provide machine instructions and/or data to a programmable processor.

To provide for interaction with a user, the systems and techniques described here can be implemented on a computer having a display device (e.g., a CRT (cathode ray tube) or LCD (liquid crystal display) monitor) for displaying information to the user and a keyboard and a pointing device (e.g., a mouse or a trackball) by which the user can provide input to the computer. Other kinds of devices can be used to provide for interaction with a user as well; for example, feedback provided to the user can be any form of sensory feedback (e.g., visual feedback, auditory feedback, or tactile feedback); and input from the user can be received in any form, including acoustic, speech, or tactile input.

The systems and techniques described here can be implemented in a computing system that includes a back end component (e.g., as a data server), or that includes a middleware component (e.g., an application server), or that includes a front end component (e.g., a client computer having a graphical user interface or a Web browser through which a user can interact with an implementation of the systems and techniques described here), or any combination of such back end, middleware, or front end components. The components of the system can be interconnected by any form or medium of digital data communication (e.g., a communication network). Examples of communication networks include a local area network (LAN), a wide area network (WAN), and the Internet.

The computing system can include clients and servers. A client and server are generally remote from each other and typically interact through a communication network. The relationship of client and server arises by virtue of computer programs running on the respective computers and having a client-server relationship to each other.

Systems and methods described herein have been described generally, with specific examples of use for intraluminal/vascular characterization. However, the disclosure is not limited to those specific examples; other applications are also contemplated. In general, multimodal characterization systems and/or methods as disclosed herein may be used for characterization of any sample desired. In some embodiments, a sample is in vivo. In some embodiments, a sample is in vitro. In some embodiments, a sample is a portion of a living subject (e.g., in vivo or resected), such as a human. In some embodiments, a sample is not living. For example, various pipes, tunnels, tubes, or the like can be characterized using systems and methods disclosed herein. In some embodiments, first sample data from a first characterization modality and second sample data from a second characterization modality are vascular data and/or intraluminal data. For example, OCT data and diffuse spectroscopy data from a cardiac imaging catheter system used to characterize a patient's artery are vascular intraluminal data. Characterization modalities may be used to acquire data in any suitable fashion, for example a catheter system will generally (though not necessarily) acquire data from characterization modalities during a pullback. Data may be acquired prior to pullback [e.g., to determine when to initiate (e.g., automatically) pullback].

At least part of the methods, systems, and techniques described in this specification may be controlled by executing, on one or more processing devices, instructions that are stored on one or more non-transitory machine-readable storage media. Examples of non-transitory machine-readable storage media include read-only memory, an optical disk drive, memory disk drive, and random access memory. At least part of the methods, systems, and techniques described in this specification may be controlled using a computing system comprised of one or more processing devices and memory storing instructions that are executable by the one or more processing devices to perform various control operations.

In this application, unless otherwise clear from context or otherwise explicitly stated, (i) the term “a” may be understood to mean “at least one”; (ii) the term “or” may be understood to mean “and/or”; and (iii) the terms “comprising” and “including” may be understood to encompass itemized components or steps whether presented by themselves or together with one or more additional components or steps.

At least part of the methods, systems, and techniques described in this specification may be controlled by executing, on one or more processing devices, instructions that are stored on one or more non-transitory machine-readable storage media. Examples of non-transitory machine-readable storage media include read-only memory, an optical disk drive, memory disk drive, and random access memory. At least part of the methods, systems, and techniques described in this specification may be controlled using a computing system comprised of one or more processing devices and memory storing instructions that are executable by the one or more processing devices to perform various control operations.

It is contemplated that systems, devices, methods, and processes of the disclosure encompass variations and adaptations developed using information from the embodiments described herein. Adaptation and/or modification of the systems, devices, methods, and processes described herein may be performed by those of ordinary skill in the relevant art.

Throughout the description, where articles, devices, and systems are described as having, including, or comprising specific components, or where processes and methods are described as having, including, or comprising specific steps, it is contemplated that, additionally, there are articles, devices, and systems according to certain embodiments of the present disclosure that consist essentially of, or consist of, the recited components, and that there are processes and methods according to certain embodiments of the present disclosure that consist essentially of, or consist of, the recited processing steps.

It should be understood that the order of steps or order for performing certain action is immaterial so long as operability is not lost. Moreover, two or more steps or actions may be conducted simultaneously. As is understood by those skilled in the art, the terms “over”, “under”, “above”, “below”, “beneath”, and “on” are relative terms and can be interchanged in reference to different orientations of the layers, elements, and substrates included in the present disclosure. For example, a first layer on a second layer, in some embodiments means a first layer directly on and in contact with a second layer. In other embodiments, a first layer on a second layer can include another layer there between.

Certain embodiments of the present disclosure were described above. It is, however, expressly noted that the present disclosure is not limited to those embodiments, but rather the intention is that additions and modifications to what was expressly described in the present disclosure are also included within the scope of the disclosure. Moreover, it is to be understood that the features of the various embodiments described in the present disclosure were not mutually exclusive and can exist in various combinations and permutations, even if such combinations or permutations were not made express, without departing from the spirit and scope of the disclosure. The disclosure has been described in detail with particular reference to certain embodiments thereof, but it will be understood that variations and modifications can be effected within the spirit and scope of the claimed invention.

What is claimed is:

1. A method for detecting a feature of interest, the method comprising:
receiving, by a processor of a computing device, first sample data from a first characterization modality and second sample data from a second characterization modality;
detecting, by the processor, a feature of interest [e.g., a structure external or internal to a subject (e.g., a physiological structure)] by providing the first sample data and the second sample to a machine-learned algorithm; and
outputting (e.g., from the algorithm), by the processor, a feature representation of the feature of interest that is oriented with respect to the first characterization modality.

2. The method of any one of the preceding claims, wherein the machine-learned algorithm has been trained to detect two or more features of interest.

3. The method of any one of the preceding claims, wherein the first sample data and the second sample data are both from intraluminal characterization modalities.

4. The method of any one of the preceding claims, wherein the first characterization modality is an interferometry modality (e.g., OCT) and the second characterization modality is an intensity measurement (e.g., a fluorescence modality).

5. The method of any one of the preceding claims, wherein the first characterization modality is a depth-dependent imaging modality (e.g., OCT) and the second characterization modality is a wavelength-dependent measurement modality (e.g., NIRS).

6. The method of any one of the preceding claims, wherein one or both of the first characterization modality and the second characterization modality are processed (e.g., formatted) prior to being input to the machine-learned algorithm.

7. The method of any one of the preceding claims, wherein one or both of the first characterization modality and the second characterization modality have been registered (e.g., to one another) prior to being input to the machine-learned algorithm.

8. The method of any one of the preceding claims, comprising registering, by the processor, one or both of the first sample data and the second sample data (e.g., to one another) prior to inputting the first sample data and the second sample data to the machine-learned algorithm.

9. The method of any one of the preceding claims, wherein the machine-learned algorithm has been trained to detect features using labels from either only the first characterization modality or the second characterization modality.

10. The method of any one of the preceding claims, wherein the machine-learned algorithm outputs detected features in reference to the first characterization modality (e.g., only the first characterization modality).

11. The method of any one of the preceding claims, wherein the first sample data are generated from the first characterization modality detected at a first region having a first tissue volume within a bodily lumen and the second sample data are generated from the second characterization modality detected at a second region having a second volume within a bodily lumen.

12. The method of claim 10, wherein an intraluminal characterization volume of each modality does not completely overlap.

13. The method of claim 11, wherein the first region and the second region do not completely overlap.

14. The method of any one of the preceding claims, wherein the first sample data are generated from detection with the first characterization modality at a time t₁ and the second sample data are generated from detection with the second characterization modality at time t₂, where t₂−t₁<1 ms.

15. The method of any one of the preceding claims, wherein the first sample data and the second sample data are combined into a combined sample data and the combined sample data are input into the machine-learned algorithm during the detecting step.

16. The method of claim 15, wherein the combining of the first sample data and the second sample data comprises (e.g., consists of) appending the first sample data to the second sample data.

17. The method of claim 16, wherein the appending comprises merging the first sample data and the second sample data together.

18. The method of any one of the preceding claims, wherein the machine-learned algorithm has multiple stages where data can be input.

19. The method of claim 18, wherein information from the first characterization modality and from the second characterization modality are input to the machine-learned algorithm as two unique inputs at different stages.

20. The method of claim 18 or claim 19, wherein the first sample data and the second sample data are separately input to the machine-learned algorithm at different ones of the multiple stages.

21. The method of any one of the preceding claims, wherein each sample data undergoes feature extraction, and outputs of the feature extraction are used as inputs to the machine-learned algorithm.

22. The method of any one of the preceding claims, comprising:
inputting, by the processor, the first sample data and the second sample data to one or more feature extractors; and
generating, by the processor, outputs from the one or more feature extractors, wherein the detecting comprises inputting the outputs from the one or more feature extractors into the machine-learned algorithm.

23. The method of any one of the preceding claims, comprising, segmenting and classifying, by the processor, one or more foreign objects (e.g., one or more fiber optics, one or more sheaths, one or more stent struts, one or more balloons).

24. The method of claim 23, comprising segmenting and classifying, by the processor, the one or more foreign objects with the machine-learned algorithm (e.g., using the first sample data and the second sample data as inputs to the algorithm) (e.g., wherein the detecting step comprises the segmenting and classifying).

25. The method of any one of the preceding claims, comprising segmenting and classifying, by the processor, one or more vascular structures (e.g., lumen, intima, medial, external elastic membrane, branching).

26. The method of claim 25, comprising segmenting and classifying, by the processor, the one or more vascular structures with the machine-learned algorithm (e.g., using the first sample data and the second sample data as inputs to the algorithm) (e.g., wherein the detecting step comprises the segmenting and classifying).

27. The method of any one of the preceding claims, comprising segmenting and classifying, by the processor, plaque morphology based on contents of the plaque (e.g., calcium, macrophages, lipids, collagen or fibrous tissue).

28. The method of claim 27, comprising segmenting and classifying, by the processor, the plaque morphology with the machine-learned algorithm (e.g., using the first sample data and the second sample data as inputs to the algorithm) (e.g., wherein the detecting step comprises the segmenting and classifying).

29. The method of any one of the preceding claims, comprising segmenting and classifying, by the processor, one or more necrotic cores or thin-cap fibroatheroma (TCFA).

30. The method of claim 29, comprising segmenting and classifying, by the processor, the one or more necrotic cores or TCFA with the machine-learned algorithm (e.g., using the first sample data and the second sample data as inputs to the algorithm) (e.g., wherein the detecting step comprises the segmenting and classifying).

31. The method of any one of the preceding claims, comprising detecting (e.g., segmenting and classifying), by the processor, one or more arterial pathologies.

32. The method of claim 31, comprising detecting (e.g., segmenting and classifying), by the processor, the one or more arterial pathologies with the machine-learned algorithm (e.g., using the first sample data and the second sample data as inputs to the algorithm) (e.g., wherein the detecting step comprises the segmenting and classifying).

33. The method of any one of the preceding claims, comprising detecting (e.g., segmenting and classifying), by the processor, one or more spectroscopy-sensitive markers.

34. The method of claim 33, comprising detecting (e.g., segmenting and classifying), by the processor, the one or more spectroscopy-sensitive markers (e.g., one or more fiducials) with the machine-learned algorithm (e.g., using the first sample data and/or the second sample data as inputs to the algorithm) (e.g., wherein the detecting step comprises the segmenting and classifying).

35. The method of claim 33 or claim 34, comprising adjusting (e.g., correcting), by the processor, an image (e.g., an image produced by an interference technique, e.g., an OCT image) based on the detection of the one or more spectroscopy-sensitive markers (e.g., thereby reducing effect of non-uniform rotational distortions of a probe (e.g., imaging catheter) observable in the image).

36. The method of any one of the preceding claims, wherein the feature of interest comprises (e.g., is) one or more foreign objects, one or more vascular structures, plaque morphology, one or more arterial pathologies, one or more necrotic cores or thin-cap fibroatheroma, or any combination thereof.

37. The method of any one of the preceding claim, wherein detecting, by the processor, the feature of interest comprises segmenting and classifying the feature of interest.

38. The method of any one of the preceding claims, comprising determining, by the processor, one or more measurements based on the feature of interest (e.g., using the machine-learned algorithm).

39. The method of claim 38, wherein the one or more measurements comprise a geometric measurement (e.g., angle, thickness, distance).

40. The method of claim 38 or claim 39, wherein the one or more measurements comprises an image-based measurement (e.g., contrast, brightness, histogram).

41. The method of any one of the preceding claims, comprising determining, by the processor, a bad frame, insufficient blood flushing, contrast injection detection, or a combination thereof with the machine-learned algorithm (e.g., using the first sample data and the second sample data as inputs to the algorithm).

42. The method of any one of the preceding claims, comprising automatically (e.g., by the processor) initiating pullback of an imaging catheter and/or scanning of a probe based on the feature of interest detected with the machine-learned algorithm.

43. The method of any one of the preceding claims, comprising determining, by the processor, an optical probe break based on the feature of interest detected with the machine-learned algorithm.

44. The method of any one of the preceding claims, comprising detecting, by the processor, poor transmission based on the feature of interest detected with the machine-learned algorithm.

45. The method of any one of the preceding claims, comprising correcting, by the processor, an image (e.g., an image produced by an interference technique, e.g., an OCT image) based on the feature of interest detected with the machine-learned algorithm (e.g., thereby reducing effects of non-uniform rotational distortions of a probe (e.g., imaging catheter) observable in the image).

46. The method of any one of the preceding claims, comprising generating the first sample data using the first characterization modality and the second sample data using the second characterization modality.

47. The method of claim 46, wherein generating the first sample data and the second sample data comprises performing a catheter pullback.

48. The method of any one of the preceding claims, comprising enhancing, by the processor, the feature representation with respect to the first characterization modality based on the second sample data and/or with respect to the second characterization modality based on the first sample data (e.g., automatically with the machine-learned algorithm) (e.g., wherein the enhanced feature representation is output from the machine-learned algorithm).

49. The method of any one of the preceding claims, wherein the feature representation is registered to the first sample data, the second sample data, or both the first sample data and the second sample data.

50. The method of any one of the preceding claims, comprising outputting (e.g., displaying), by the processor, the feature representation overlaid over an image (e.g., OCT image) derived from the first sample data or the second sample data.

51. The method of any one of the preceding claims, wherein the method is performed after pullback of a catheter with which the first sample data and the second sample data are acquired (e.g., automatically upon completion of the pullback).

52. The method of any one of the preceding claims, wherein the feature representation of the feature of interest that is oriented also with respect to the second characterization modality.

53. A multimodal system for feature detection, the system comprising:
a processor; and
a non-transitory computer readable medium having instructions stored thereon that when executed by the processor automatically upon initiation of a characterization session, cause the processor to:
receive, by the processor a first sample data from a first characterization modality and second sample data from a second characterization modality;
detect, by the processor, a feature of interest by providing the first sample data and the second sample to a machine-learned algorithm; and
output, by the processor, a feature representation of the feature of interest that is oriented with respect to the first characterization modality.

54. The system of claim 53, further comprising a display.

55. The system of claim 54, wherein the instructions, when executed by the processor automatically upon initiation of the characterization session, cause the processor to output the feature representation with the display.

56. The system of any one of claims 53-55, further comprising a first characterization subsystem for the first characterization modality and a second characterization subsystem for the second characterization modality.

57. The system of any one of claims 53-56, wherein the instructions, when executed by the processor automatically upon initiation of the characterization session, cause the processor to perform the method of any one of claims 1-48.

58. A method for measuring a feature of interest, the method comprising:
receiving, by a processor of a computing device, first sample data from a first characterization modality and second sample data from a second characterization modality;
detecting, by the processor, a feature of interest by providing the first sample data and the second sample data to a machine-learned algorithm; and
determining, by the processor, one or more measurements of a feature representation of the feature of interest.

59. The method of claim 58, further comprising displaying, by the processor, the measurement on a display.

60. The method of claim 58 or claim 59, wherein the first sample data and the second sample data are both from intraluminal characterization modalities.

61. The method of any one of claims 58-60, wherein the measurement comprises a geometric measurement (e.g., angle, thickness, distance, depth).

62. The method of any one of claims 58-61, wherein the measurement comprises an image-based measurement (e.g., contrast, brightness, histogram).

63. The method of any one of claims 58-62, wherein the measurement quantifies an aspect of a foreign object within a lumen (e.g., one or more fiber optics, one or more sheaths, one or more stent struts, one or more balloons).

64. The method of any one of claims 58-63, wherein the measurement relates to positioning of a foreign object within a lumen (e.g., stent placement within an artery).

65. The method of any one of claims 58-64, wherein the measurement quantifies an aspect of a vascular structure (e.g., of a lumen, an intima, a medial, an external elastic membrane, branching).

66. The method of any one of claims 58-65, wherein the measurement quantifies an aspect of plaque morphology (e.g., quantity of calcium, macrophage, lipid, fibrous tissue, or necrotic core within an area).

67. The method of any one of claims 58-66, wherein the measurement quantifies risk associated with a detected plaque (e.g., a detected TCFA).

68. The method of any one of claims 58-67, wherein the measurement comprises a cap thickness over a lipid pool or necrotic core.

69. The method of any one of claims 58-68, wherein the measurement comprises a plaque burden or lipid core burden (e.g., max burden over a distance).

70. The method of any one of claims 58-69, wherein the measurement comprises plaque vulnerability.

71. The method of any one of claims 58-70, wherein the measurement comprises a calcium measurement (e.g., arc, thickness, extent, area, volume, ratio of calcium to other).

72. The method of any one of claims 58-71, wherein the measurement comprises a lipid measurement (e.g., arc, thickness, extent, area, volume, ratio of lipid to other).

73. The method of any one of claims 58-72, wherein the measurement comprises stent malapposition, stent length, or stent location planning.

74. The method of claim 73, comprising automatically determining, by the processor, (e.g., with one or more machine-learned algorithms, e.g., the machine-learned algorithm) a location for a stent placement based on the one or more measurements (e.g., by optimization).

75. The method of any one of claims 58-74, wherein the one or more measurements comprises lumen area.

76. The method of any one of claims 58-75, wherein the measurement comprises a measurement on an external elastic membrane or external elastic lamina.

77. The method of any one of claims 58-76, wherein the machine-learned algorithm outputs the measurement.

78. The method of any one of claims 58-77, comprising generating the first sample data using the first characterization modality and the second sample data using the second characterization modality.

79. The method of claim 78, wherein generating the first sample data and the second sample data comprises performing a catheter pullback.

80. A method for enhancing data acquired from a bodily lumen, the method comprising:
receiving, by a processor of a computing device, first sample data from a first characterization modality and second sample data from a second characterization modality;
detecting, by the processor, a feature of interest by providing the first sample data and the second sample to a machine-learned algorithm; and
outputting from the algorithm, by the processor, a transformed representation of either the first sample data or the second sample data, or both, based on the detected feature.

81. The method of claim 80, comprising displaying (e.g., by the processor) the transformed representation (e.g., wherein the outputting comprises displaying the transformed representation).

82. The method of claim 80 or claim 81, comprising inputting, by the processor, the transformed representation into another machine-learned algorithm for feature detection.

83. The method of claim 82, comprising detecting, by the processor, a feature of interest with the machine-learned algorithm for feature detection based on the transformed representation input.

84. The method of any one of claims 80-83, wherein the transformed representation is an enhanced OCT image.

85. The method of claim 84, wherein the transformed representation corrects for non-uniform rotational distortions of a probe using the detected feature.

86. The method of any one of claims 80-83, wherein the transformed representation is enhanced reflectance data.

87. The method of any one of claims 80-83, wherein the transformed representation is enhanced spectroscopy data.

88. The method of any one of claims 80-87, wherein the transformed representation is in a new image space or color scheme.

89. The method of any one of claims 80-88, comprising determining, by the processor, a specular versus diffuse reflection ratio based on one of the first sample data and the second sample data (e.g., with the machine-learned algorithm) and improving or enhancing attenuation correction in the transformed representation, wherein the transformed representation is of either the other of the first sample data and the second sample data.

90. The method of any one of claims 80-89, wherein the transformed representation is an attenuation corrected representation.

91. A method for detecting a feature of interest, the method comprising:
receiving, by a processor of a computing device, first sample data from a first characterization modality and second sample data from a second characterization modality; and
detecting, by the processor, one or more features of interest by providing the first sample data and the second sample to a machine-learned algorithm.

92. The method of claim 91, comprising automatically (e.g., by the processor) initiating (i) pullback of an imaging catheter and/or (ii) scanning of a probe based on the one or more features of interest detected with the machine-learned algorithm.

93. A method for compensating for non-uniform rotational distortions (NURD), the method comprising:
receiving, by a processor of a computing device, first sample data from a first characterization modality and second sample data from a second characterization modality;
evaluating, by the processor, NURD by providing at least one of the first sample data and the second sample to a machine-learned algorithm; and
correcting, by the processor, (e.g., with the machine-learned algorithm) at least one of the first sample data and the second sample data based on the evaluating to accommodate for NURD.

94. The method of claim 93, wherein the evaluating comprises providing only the first sample data to the machine-learned algorithm and the correcting is of the second sample data.

95. The method of claim 93 or claim 94, wherein the first sample data and/or the second sample data are an image.

96. A method for determining improved physiological measurements, the method comprising:
receiving, by a processor, first sample data from a first characterization modality;
receiving, by the processor, information about a feature of interest (e.g., a location and/or composition of the feature of interest), wherein at least a portion of the first sample data corresponds to the feature of interest (e.g., a feature representation of the feature of interest is comprised in the first sample data); and
determining, by the processor, a physiological measurement using the first sample data and the information.

97. The method of claim 96, wherein the feature of interest is a plaque or a curvature of a vessel (e.g., an artery).

98. The method of claim 96 or claim 97, wherein the physiological measurement corresponds to a flow, a pressure drop, or a resistance for a vascular structure (e.g., artery) (e.g., wherein the physiological measurement is a measurement of flow rate, fractional flow reserve, pressure drop, absolute or relative coronary flow (CF), fractional flow reserve (FFR), instantaneous wave free ratio/resting full cycle ratio (iFR/RFR), index of microcirculatory resistance (IMR), hyperemic microvascular resistance (HMR), hyperemic stenosis resistance (HSR), coronary flow reserve (CFR), or a combination thereof).

99. The method of any one of claims 96-98, wherein the first characterization modality is an interferometric modality (e.g., OCT).

100. The method of any one of claims 96-99, comprising determining, by the processor, the location and/or composition of the feature of interest using second sample data from a second characterization modality (e.g., and also the first sample data).

101. The method of claim 100, wherein the second characterization modality is a spectroscopic modality (e.g., NIRS).

102. The method of any one of claims 96-101, comprising determining, by the processor, the information about the feature of interest using the first sample data.

103. The method of any one of claims 96-102, comprising determining, by the processor, the information about the feature of interest using a machine-learned algorithm [e.g., by providing the first sample data (e.g., and/or the second sample data) to the machine-learned algorithm].

104. The method of any one of claims 96-103, comprising detecting, by the processor, the feature of interest using a (e.g., the) machine-learned algorithm [e.g., by providing the first sample data (e.g., and/or the second sample data) to the machine-learned algorithm].

105. A method for making physiological measurements, the method comprising:
receiving, by a processor of a computing device, first sample data from a first characterization modality and second sample data from a second characterization modality; and
determining, by the processor, a physiological measurement by providing the first sample data and the second sample data to a machine-learned algorithm.

106. A method of training a machine-learned algorithm, the method comprising:
providing, by a processor of a computing device, training data to a machine-learning algorithm, wherein the training data is labelled with training labels that have been derived from data from a second characterization modality different from the first characterization modality.

107. The method of claim 106, wherein the first characterization modality is an interferometric modality (e.g., OCT) and the second characterization modality is a spectroscopic modality (e.g., NIRS).

108. The method of claim 106 or claim 107, wherein the training data does not comprise data from the second characterization modality.

109. A method for detecting a feature of interest and/or determining a measurement thereof, the method comprising:
receiving, by a processor of a computing device, first sample data from a first characterization modality [e.g., an interferometric modality (e.g., OCT)];
detecting, by the processor, a feature of interest by providing the first sample data to a machine-learned algorithm that has been trained on training data from the first characterization modality, the training data labelled with training labels derived from data from a second characterization modality [e.g., a spectroscopic modality (e.g., NIRS)]; and
outputting (e.g., from the algorithm), by the processor, (i) a feature representation of the feature of interest that is oriented with respect to at least the first characterization modality, (ii) one or more measurements (e.g., of the feature representation and/or comprising a physiological measurement), or (iii) both (i) and (ii).

110. The method of claim 109, wherein the machine-learned algorithm does not accept data from the second characterization modality as input.

111. The method of claim 109 or claim 110, wherein the machine-learned algorithm accepts data from no other characterization modality than the first characterization modality as input.

112. The method of any one of claims 109-111, comprising outputting, by the processor, the feature representation and/or at least one measurement of the feature representation, wherein the feature of interest is a plaque or portion thereof (e.g., lipid core of the plaque).

113. A system, the system comprising:
a processor; and
a non-transitory computer readable medium having instructions stored thereon that when executed by the processor (e.g., automatically upon initiation of a characterization session), cause the processor to perform the method of any one of claims 1-52 and 58-112.
